SEQUENCE LISTING (7 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

SEQ ID NO: 3 - DNA, 31 bp, Artificial Sequence
  Description of Artificial Sequence AB40/42 Forward Primer
tcactcgagaatggatgcagaattccgacat

SEQ ID NO: 4 - DNA, 29 bp, Artificial Sequence
  Description of Artificial Sequence AB42 Reverse Primer
atggatccttacgctatgacaacaccgaa

SEQ ID NO: 5 - DNA, 29 bp, Artificial Sequence
  Description of Artificial Sequence AB40 Reverse Primer
tcgatccttagacaacaccgcccaccatg

SEQ ID NO: 6 - DNA, 27 bp, Artificial Sequence
  Description of Artificial Sequence AB-SP1 forward primer
ttactcgagatgctgcccggtttggca

SEQ ID NO: 7 - DNA, 34 bp, Artificial Sequence
  Description of Artificial Sequence AB-SP2 reverse primer
ggaattctgcatccatcgcccgagccgtccaggc

(19) United States
(12) Patent Application Publication
(10) Pub. No.: US 2004/0248766 A1
(43) Pub. Date: Dec. 9, 2004

(54) Selective inhibition of intracellular amyloid-beta neurotoxicity in human neurons

(76) Inventor: Andrea LeBlanc, Chambly (CA)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/481,387

(22) PCT Filed: Jun. 18, 2002
(86) PCT No.: PCT/CA02/00916

Related U.S. Application Data
(60) Provisional application No. 60/298,373, filed on Jun. 18, 2001.
(60) Provisional application No. 60/330,543, filed on Oct. 24, 2001.

(51) Int. Cl. A61K 38/17; A61K 48/00

(52) U.S. Cl. 514/2; 514/44

(57) ABSTRACT

While the extracellular accumulation of amyloid-β in the brain parenchyma is a pathological hallmark of Alzheimer's disease, its role as a cause or a consequence of AD is still debated. As described herein, intracellular Aβ1-42 is shown to be selectively toxic to neurons. The present invention provides methods of screening for compounds for the prevention and treatment of A amyloid associated diseases such as Alzheimer's disease, Down's Syndrome, cerebral amyloid angiopathy, and inclusion body myositis.

### CROSS-REFERENCE TO RELATED APPLICATIONS

This application claims the priority of U.S. Provisional Patent Application Nos. 60/298,373, filed Jun. 18, 2001, and Ser. No. 60/330,543, filed Oct. 24, 2001, both of which are incorporated herein by reference.

### BACKGROUND OF THE INVENTION

Alzheimer's Disease (“AD”) is a progressive neurodegenerative disease of the central nervous system characterized by the presence of extracellular senile plaques, intra-neuronal neurofibrillary tangles, the loss of synapses, and neuronal cell death. The amyloid β peptide (“Aβ”) is a major component of the extracellular senile plaque.

While extensive research has been conducted on the relevance of extracellular Aβ deposits in AD (Selkoe, D. J., Trends in Cell Biology 8:447-453, 1998), little is known about the properties of intracellular Aβ. Aβ naturally arises from the metabolic processing of the amyloid precursor protein (“APP”) in the endoplasmic. reticulum (“ER”), the Golgi apparatus, or the endosomal-lysosomal pathway, and most is normally secreted as a 40 (“Aβ₁₋₄₀”) or 42 (“Aβ₁₋₄₂”) amino acid peptide (Selkoe, D. J., Annu. Rev. Cell Biol. 10:373-403, 1994).

An N-terminally truncated form of Aβ₁₋₄₂ (“Aβ₄₂”) accumulates in the ER in aging cell cultures (Greenfield, J. P., et al., Proc. Natl. Acad. Sci. U.S.A. 96:742-7, 1999; Yang, A. J., et al., J. Biol. Chem. 274:20650-6, 1999). Recently, the presence of intracellular Aβ₄₂ has been detected in the brains of individuals with AD or Down's Syndrome, in APP transgenic mice, and aging monkeys.

In AD brains, the intracellular Aβ₄₂ accumulates as aggregates or granules in the cytoplasm of neurons (D'Andrea, M. R., et al., Histopathology 38:120-34, 2001; and Gouras, G. K., et al., Am. J. Pathol. 156:15-20, 2000). The accumulation of Aβ precedes the appearance of neurofibrillary tangles (“NFT”) and senile plaques and is observed in regions affected early in Alzheimer's Disease, the hippocampus and entorhinal cortex. The intracellular Aβ does not appear to be fibrillar since it is not stained by Bielchowsky silver stain, Thioflavin S, or Congo Red, nor does it require formic acid treatment for imnmunostaining.

In Down's Syndrome brains, accumulation of intracellular Aβ₁₋₂₈ and Aβ₄₀ precedes Aβ₄₂ and these intracellular Aβ accumulations precede the appearance of diffluse plaques, senile core plaques, and NFT formation (Gyure, K. A., et al., Arch. Pathol Lab. Med. 125:489-92, 2001). In aging monkeys, non-fibrillar neuronal and non-neuronal intracellular Aβ precedes the deposition of extracellular and fibrillar Aβ (Martin, L. J., et al., Am. J. Pathol. 145:1358-1381, 1994b).

Furthermore, the accumulation of intracellular Aβ also precedes plaque formation in mutant APP/PS-1 transgenic mice (Wirths, O., et al., Neurosci. Lett. 306:116-20, 2001). Intracellular Aβ also accumulates in the APPV717F mutation where synaptic loss precedes extracellular Aβ deposition (Hsia, A. Y., et al., Proc Natl. Acad. Sci. U.S.A. 96:3228-33, 1999; Li, Q. X., et al., J. Neurochem. 72:2479-87, 1999; and Masliah, E., et al., J. Neurosci. 16:5795-811, 1996), and intracellular Aβ is associated with neuronal loss in PS-1 transgenic mice in the absence of extracellular Aβ deposition (Chui, D. H., et al., Nat. Med. 5:560-4, 1999).

A role for Aβ as a primary cause for AD is supported by the presence of extracbllular amyloid β peptide (“Aβ”) deposits in senile plaques of Alzheimer's disease (“AD”), the increased production of Aβ in cells harboring mutant AD associated genes, e.g., amyloid precursor protein, presenilin I and presenilin II; and the toxicity of extracellular fibrillar Aβ to cells in culture (reviewed by Selkoe, D. J., Trends in Cell Biology 8, 447-453 (1998)).

A conventional view is that the extracellular Aβ deposits are a tombstone of AD (Glabe, C., Nat. Med. 6, 133-4 (2000)). Arguments that support this view are that senile plaques do not necessarily correlate with the level of cognitive impairment in AD patients, the presence of high levels of extracellular Aβ in transgenic mouse models fails to induce all of the pathological hallmarks of AD, such as synaptic degeneration, neurofibrillary tangles, and neuronal loss; and the toxicity of Aβ peptide in cell cultures and brains is limited to high non-physiological doses of the peptide.

### SUMMARY OF THE INETION

Extracellular Aβ is a major target for the development of therapeutics for Aβ-related diseases such as Alzheimer's disease and CAA. Several strategies to inhibit Aβ-fibril formation are being considered, (reviewed in Gervais, F., European Biopharmaceutical Review, 40-42, Autumn 2001; May, P.C., DDT, 6:459-462, 2001). These approaches center around the prevention of extracellular Aβ fibril formation, and are based on the observation that Aβ forms large extracellular amyloid deposits which are associated with disease. Exemplary strategies, such as described herein, include: 1) Suppression of APP expression which would preclude the production of Aβ peptide; 2) Suppression of Aβ production by inhibiting APP processing, e.g., inhibiting β-secretase or γ-secretase, in order to block or reduce Aβ production; 3) Inhibition of Aβ fibrillogenesis by interfering with Aβ self-association or by interfering with Aβ association with molecular chaperones such as HSPG, e.g., using peptides or compounds which mimic the anionic property of glycosamninoglycans; 4) Inhibition of the neurodegenerative effect of Aβ; and 5) Increasing Aβ clearance from the CNS to the peripheral system, e.g., using a therapeutic vaccine approach.

In contrast, the use of soluble intracellular or cytosolic Aβ as a target represents a novel approach to the development of therapeutics for Aβ-related disorders. Indeed, it was heretofore unknown that intracellular Aβ is toxic to neurons.

The present invention relates to a new method for screening for drugs against disease states associated with amyloidosis, such as Alzheimer's disease, and for treating or preventing disease states associated with amyloidosis, such as Alzheimer's disease.

In a preferred embodiment, the method is used to treat Alzheimer's disease (e.g. sporadic or familial AD). The method can also be used prophylactically or therapeutically to treat other clinical occurrences of amyloid-β deposition, such as in Down's syndrome individuals and in patients with cerebral amyloid angiopathy (“CAA”) or hereditary cerebral hemorrhage.

CAA remains a largely untreatable disease often not diagnosed until autopsy. The term CAA refers to the specific deposition of amyloid fibrils in the walls of leptomingeal and cortical arteries, arterioles and in capillaries and veins. It is commonly associated with Alzheimer's disease, Down's syndrome, and normal aging, as well as with a variety of familial conditions related to stroke and/or dementia (see, Frangione et al., Amyloid: J. Protein Folding Disord. 8(Suppl. 1):3642, 2001). It ranges in severity from asymptomatic amyloid deposition in otherwise normal cerebral vessels to complete replacement and breakdown of the cerebrovascular wall.

Severe CAA can cause lobar cerebral hemorrhage, transient neurologic symptoms, and dementia with leukoencephalopathy (see, Greenberg, Neurology 51:690-694, 1998). Advanced cases of CAA demonstrate structural changes to the walls of the amytoid-laden vessel such as cracking between layers, smooth muscle cell toxicity, microaneuryism formation, and fibrinoid necrosis.

CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia.

Additionally, abnormal accumulation of APP and of amyloid-β peptide in muscle fibers has been implicated in the pathology of sporadic inclusion body myositis (“IBM”) (Askanas, V., et al., Proc. Natl. Acad. Sci. USA 93:1314-1319, 1996; Askanas, V., et al., Current Opinion in Rheumatology 7:486-496, 1995).

Accordingly, the compounds identified by the methods of the invention may be used prophylactically or therapeutically in the treatment of disorders in which amyloid-beta peptide is abnormally deposited at non-neurological locations, such as treatment of IBM by delivery of the compounds to muscle fibers.

One aim of the present invention is to provide a method for treating or preventing Alzheimer's disease, cerebral amyloid angiopathy, Down's Syndrome, or inclusion body myositis.

Another aim of the present invention is to provide a new method for screening for drugs against Alzheimer's disease, cerebral amyloid angiopathy, Down's Syndrome, or inclusion body myositis.

In accordance with the present invention, there is provided a method for treating or preventing a disease state associated with amyloidosis, said method comprising administering to a subject a therapeutically effective amount of a compound for reducing the intracellular concentration of Aβ, such that said disease state associated with amyloidosis is treated or prevented. Preferably, the disease state is intracellular amyloid production or accumulation associated with Alzheimer's disease, cerebral amyloid angiopathy, Down's Syndrome, or inclusion body myositis.

In one embodiment of the invention, the compound is an intracellular protease capable of eliminating Aβ, or preventing accumulation of Aβ. The expression of the protease may be induced in neurons of a subject upon administration of a chemical substance (e.g., a drug).

In accordance with the present invention, there is also provided a transfected cell capable of expressing an agent capable of inducing in neurons an intracellular protease capable of eliminating Aβ or preventing accumulation of Aβ, said cell being incapable of expressing said protease when not so transfected.

The present invention may thus be used for gene therapy for Alzheimer's disease, wherein such therapy comprises a step of administering an expression vector to a subject suffering from Alzheimer's disease, said vector coding under suitable conditions for an agent capable of inducing the espression in neurons of an intracellular protease or a ribozyme capable of eliminating Aβ or preventing accumulation of Aβ, or antisense gene therapy.

Still in accordance with the present invention, there is also provided a method for preventing or inhibiting amyloid production in a subject, said method comprising administering to a subject a therapeutically effective amount of a compound capable of reducing the intracellular concentration of Aβ, such that amyloid β production or accumulation, intracellular or extracellular, is prevented or inhibited.

Further in accordance with the present invention, there is also provided a method for preventing or inhibiting amyloid production in a subject, said method comprising administering to a subject a therapeutically effective amount of a compound capable of inhibiting Aβ intracellular formation, such that amyloid β production or accumulation is prevented or inhibited.

The present invention also provides a method for modulating amyloid-associated damage to cells, comprising the step of administering a compound capable of reducing the intracellular concentration of Aβ, such that said amyloid-associated damage to cells is modulated. In preferred aspects of the invention, the methods for modulating amyloid-associated damage to cells comprise a step of administering a compound capable of reducing the intracellular concentration of Aβ₁₋₄₂.

In accordance with the present invention, there is furrer provided a method for preventing or inhibiting amyloid aggregation in a subject, said method comprising administering to a subject a therapeutically effective amount of a compound capable of inhibiting β-secretase or γ-secretase, such that intracellular amyloid β accumulation is prevented or inhibited.

In accordance with the present invention, there is further provided a method for preventing cell death in a subject, said method comprising administering to a subject a therapeutically effective amount of a compound capable of preventing intracellular Aβ-mediated events that lead to cell death.

Also in accordance with the present invention, there is provided a method for screening a potentially useful compound for treating Alzheimer's disease, cerebral amyloid angiopathy, Down's Syndrome, or inclusion body myositis, said method comprising a step of applying to a cell a compound to be screened for inhibition of intracellular Aβ-mediated (preferably Aβ₁₋₄₂-mediated) cell death and measuring an intracellular concentration of Aβ, wherein an intracellular concentration of Aβ lower than a concentration of Aβ measured for a normal cell is indicative of said compound being useful for treating Alzheimer's disease, cerebral amyloid angiopathy, Down's Syndrome, or inclusion body myositis.

Also in accordance with the present invention, there is provided a method for screening a potentially useful compound for treating Alzheimer's disease, cerebral amyloid angiopathy, Down's Syndrome, or inclusion body myositis, said method comprising a step of applying to a cell a compound to be screened for inhibition of intracellular Aβ-mediated (preferably Aβ₁₋₄₂-mediated) cell death, wherein prevention or reduction of Aβ-mediated cell death is indicative of said compound being useful for treating Alzheimer's disease, cerebral amyloid angiopathy, Down's Syndrome, or inclusion body myositis.

In accordance with the present invention, there is also provided a method for treating or preventing Alzheimer's disease, cerebral amyloid angiopathy, Down's Syndrome, or inclusion body myositis, said method comprising a step of brealing down (e.g., metabolizing) intracellular Aβ or causing excretion of Aβ in order to reduce the intracellular concentration of Aβ, thereby treating or preventing Alzheimer's disease, cerebral amyloid angiopathy, Down's Syndrome, or inclusion body myositis.

Without intending to be bound by theory, there are at least three mechanisms according to the invention in which the toxic effects of intracellular Aβ may be prevented: One is to inhibit the formation of Aβ, e.g. by using β- or γ-secretase inhibitors. Another is to activate intracellular proteases that would eliminate the Aβ and prevent its intracellular accumulation. Lastly, one is to prevent the Aβ-mediated events that lead to cell death. The methods of the invention may be carried out regardless of which mechanism is exploited. Therefore, efforts directed at inhibiting the production or accumulation of intracellular Aβ may be one of the best therapies for the treatment or prevention of early AD. The present invention provides methods for identifying drug candidates which may interfere with any of these mechanisms.

In the present application, it is shown that intracellular Aβ₁₋₄₂ but not intracellular Aβ₁₋₄₀ or intracellular Aβ₄₀₋₁ peptides is cytotoxic to human neurons, non-fibrillized Aβ₁₋₄₂ peptide is as toxic as the fibrillized peptide, and Aβ₁₋₄₂ toxicity is mediated through at least the p53 and Bax cell death pathways.

It is shown in the present invention that low levels of intracellular Aβ₁₋₄₂ selectively induce human neuronal cell death through the p53/Bax cell death pathway. Inhibition of the toxicity with actinomycin D, cycloheximide, caspase inhibitors, Bax neutralizing antibodies, and p53 DN expression indicates that cell death is of the classical programmed cell death type.

### BRIEF DESCRIPTION OF THE DRAWINGS

FIG. 1A. Intracellular Aβ neurotoxicity in primary human neurons. Fluorescent photomicrographs of microinjected neurons. Neurons were microinjected with the peptides in DTR and incubated 24 hours before staining with TUNEL for cell death or Hoechst for nuclear stain.

FIG. 1B. Aged Aβ₁₋₄₀, Aβ₁₋₄₂, Aβ₄₂₋₁, and Aβ₄₀₋₁ peptides (10 nM) were microinjected into the cytosol of human neurons and cell death was measured by TUNEL at 1, 2, 4 and 16 days after injection. Two-way ANOVAs (dfₜᵢₘₑ=4; dfₜᵣₑₐₜₘₑₙₜ=3) followed by Sheffé's test were performed to determine the statistical significance between Aβ-injected and control DTR-injected neurons. *: p<0.01.

FIG. 1C. Various doses of Aβ₁₋₄₂ and Aβ₄₀₋₁ were injected into human neurons and cell death was determined by TUNEL staining at 2, 4 or 16 days after injection. Two-way ANOVAs (dfₜᵢₘₑ=2; dfₜᵣₑₐₜₘₑₙₜ=29) followed by Sheffé's test were performed to determine the statistical significance. *: p<0.01.

FIG. 1D. Human neurons were exposed to 10 μM extracellular Aβ₁₋₄₀, Aβ₁₋₄₂ and Aβ₄₀₋₁ for 24 hours and stained with propidium iodide to reveal cellular nuclei and TUNEL to reveal cell death.

FIG. 1E. Cell death in neurons 24 hours after microinjection with pCep4β episomal cDNA constructs expressing cytosolic Aβ₁₋₄₀ and Aβ₁₋₄₂ (cAβ) or secreted Aβ₁₋₄₀ and Aβ₁₋₄₂ (sAβ). One-way ANOVA (df=5) followed by Sheffé's test determined a statistically significant difference between the Cep4β construct alone and Aβ₁₋₄₂ peptide or Cep4β-cAβ₁₋₄₂ expression construct. *p<0.01 For B-E, the data represent the mean ± SEM of 3 independent experiments.

FIG. 2A. Soluble and fibrillar Aβ₁₋₄₂ are toxic to human neurons. Electron micrographs of non-fibrillized (nf) and fibrillized (f) Aβ₁₋₄₀ and Aβ₁₋₄₂. In the Aβ₁₋₄₀ nf, the arrows point to the rare globular structures while in the Aβ₁₋₄₀ f, the arrows point to the small aligned fibrils.

FIG. 2B. Non-fibrillized or fibrillized Aβ₁₋₄₀ or Aβ₁₋₄₂ (10 nM) were injected into human neurons and neuronal cell death assessed by TUNEL staining at 24, 48 and 96 hours after injection. The data represent the mean ± SEM of 3 independent experiments. One-way ANOVA (df=14) followed by Sheffé's test were performed to determine the statistical significance between Aβ-injected and control DTR-injected neurons. *p<0.01.

FIG. 2C. Western blot analysis of fibrillized or non-fibrillized Aβ₁₋₄₀ and Aβ₁₋₄₂ with 6E10. M, D, and T represent the monomeric, dimeric and trimeric forms, respectively. A longer exposure revealed a smear also in the fibrillized Aβ₁₋₄₀ (data not shown).

FIG. 3A. Intracellular Aβ₁₋₄₂ is not toxic to primary human astrocytes, neuroblastoma, teratocarcinoma, fibroblast and kidney cell lines. Cells were microinjected with 100 nM Aβ₁₋₄₂ and DTR and incubated for 24 hours. TUNEL staining identified cell death and Hoechst staining detected nuclei.

FIG. 3B. Cell survival quantitation in several cell lines injected with DTR and Aβ₁₋₄₀, Aβ₁₋₄₂ or Aβ₄₀₋₁ and incubated 24 hours. The data represent the mean ± SEM of 3 independent experiments. One-way ANOVA (df=27) followed by Sheffé's test determined the statistical significance between Aβ-injected and control DTR-injected cells. *p<0.01.

FIG. 4. Intracellular Aβ₁₋₄₂ toxicity requires de novo protein synthesis. Neuronal cell death in non- or Aβ₁₋₄₂-injected neurons incubated in the absence (−) or presence of 5 μg/ml cycloheximide (CHX) or 5 μM actinomycin D (ACTD) for 48 hours. Neurons were pre-incubated for 1 hour in CHX and ACTD before microinjections. The data represent the mean ± SEM of 3 independent experiments. One-way ANOVA (df=5) followed by Sheffé's test determined a statistical significant difference between Aβ₁₋₄₂ in absence and presence of CHX and ACTD but not between CHX and ACTD treatment of non- and Aβ₁₋₄₂-injected neurons. *p<0.01.

FIG. 5A. Inhibition of Aβ₁₋₄₂-mediated neuronal cell death with Bcl-2 and Bax-neutralizing antibodies. Cell death in neurons co-injected with Aβ₁₋₄₂ and Bcl-2 or APP pCep4β eukaryotic cDNA expression episomal construct. *p<0.01.

FIG. 5B. Neuronal cell death in DTR alone (Control), Bax cDNA, Aβ₁₋₄₂ peptide, or recombinant active caspase-6 (R-Csp-6)-microinjected neurons in the absence (−) or presence of monoclonal Bax antibodies, 6A7 or 2D2, Bax polyclonal antisera, N-20, APP monoclonal antibody 22C11, mouse IgG or rabbit non-immune sera. Cells were incubated 24 hours after microinjections. The data represent the mean ± SEM of 3 independent experiments. One-way ANOVA (df=6, followed by Sheffé's test determined the significance of the difference between the insult in absence and presence of the antibodies. *p<0.01.

FIG. 6. Inhibition of Aβ₁₋₄₂-mediated neuronal cell death with p53 dominant negative R273H mutant but not p53 wild type. Neuronal cell death in DTR (Ctl), Aβ₁₋₄₀ peptide, Aβ₁₋₄₂ peptide, R-Csp-6, or Bax cDNA alone (−) or co-microinjected with cDNA expressing wild type (WT) or dominant negative (D)N) p53 in neurons. Microinjected cells were incubated 48 hours. The data represent the mean ± SEM of 3 independent experiments. One-way ANOVA (df=14) followed by Sheffé's test showed a statistically significant difference between Aβ₁₋₄₂ without and with p₅₃^{DN}-injected neurons. *p<0.01.

FIG. 7. Inhibition of Aβ₁₋₄₂-mediated neuronal cell death with caspase inhibitors. Neurons were pre-incubated for 1 hour in the presence of 5 μM of each inhibitor, microinjected with Aβ₁₋₄₂ peptide and incubated for 24 hours in the presence of the inhibitors before revealing cell death of injected cells with TUNEL. The data represent the mean ± SEM of 3 independent experiments. One-way ANOVA (df=11) followed by Sheffé's test showed a statistically significant difference between Aβ₁₋₄₂ and Aβ₁₋₄₂+Z-VAD-fmk, Z-VEID-fmk, and Z-IETD-fmk. *p<0.01

FIG. 8A. A histogram illustrating the relative toxicity of Aβ₁₋₄₂-mediated neuronal cell death compared with that of Aβ₂₋₄₂.

FIG. 8B. A histogram illustrating the inhibition of Aβ₁₋₄₂-mediated neuronal cell death with lithium.

### DETAILED DESCRIPTION OF THE PREFERRED EMBODIMENTS

As used herein, the term “β amyloid” refers to amyloid proteins or peptides, amyloid precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ₁₋₃₉, Aβ₁₋₄₀, Aβ₁₋₄₁, Aβ₁₋₄₂, and Aβ₁₋₄₃. For convenience of nomenclature, “Aβ₁₋₄₂” may be referred to herein simply as “Aβ₄₂” or “Aβ42” (and likewise for the other amyloid peptides discussed herein). As used herein, the terms “β amyloid,” “amyloid β,” and “Aβ” are synonymous.

The terms “disease state associated with amyloidosis” or “Aβ amyloid-associated disease or condition,” which may be used interchangeably herein, refer to any disease, condition, or disorder in which β amyloid aggregation occurs, such as Alzheimer's disease, Down's Syndrome, inclusion body myositis, and cerebral amyloid angiopathy. Clinically, amyloidosis can be primary, secondary, familial or isolated. Amyloids have been categorized by the type of amyloidogenic protein contained within the amyloid.

The term “subject” includes living organisms in which amyloidosis can occur. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate amyloid aggregation in the subject as fiurer described herein. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to modulate amyloid aggregation in the subject. Dosage regimens can be adjusted to provide the optimum therapeutic response. For example, several divided doses may be administered daily or the dose may be proportionally reduced as indicated by the exigencies of the therapeutic situation.

The term “modulating” is intended to encompass prevention or stopping of amyloid formation or accumulation, inhibition or slowing down of ftrter amyloid aggregation in a subject with ongoing amyloidosis, e.g., already having amyloid aggregates, and reducing or reversing of amyloid aggregates in a subject with ongoing amyloidosis. Modulation of amyloid aggregation is determined relative to an untreated subject or relative to the treated subject prior to treatment.

In methods of the invention, a “modulator” or a “compound capable of modulating” is preferably a small molecule having a molecular weight under 2500 Daltons, peptide, antisense oligonucleotide, antisense peptide, enzyme, antibody or fragment thereof, ribozyme, or haptomer.

The present invention pertains to a method for treating or preventing a disease state associated with amyloidosis, the method comprising administering to a subject a therapeutically effective amount of a compound for reducing the intracellular concentration of Aβ, such that said disease state associated with amyloidosisis treated or prevented. The invention also pertains to a method wherein the disease state is Aβ production or accumulation associated with Alzheimer's disease, cerebral amyloid angiopathy, Down's Syndrome, or inclusion body myositis.

In another aspect, the invention pertains to a method for treating or preventing a disease state associated with amyloidosis, the method comprising administering to a subject a therapeutically effective amount of a compound for reducing the intracellular concentration of Aβ, wherein the compound is an intracellular protease, or a compound which stimulates the endogenous production of a protease, which protease is capable of eliminating Aβ or preventing accumulation of Aβ.

The invention furrer pertains to a transfected cell capable of expressing an agent capable of inducing into neurons an intracellular protease capable of eliminating Aβ or preventing accumulation of Aβ, the cell being otherwise when not transfected (i.e., the non-transfected cell), not capable of expressing said protease.

The invention also relates to a gene therapy for treating an Aβ amyloid-associated disease or condition. Such a method includes administering an expression vector to a patient suffering from the Aβ amyloid-associated disease or condition, the vector coding under suitable conditions for an agent capable of inducing into neurons an intracellular protease capable of eliminating Aβ or preventing accumulation of Aβ.

Also, the invention relates to a method for preventing or inhibiting amyloid production in a subject. For example, such a method comprises administering to a subject a therapeutically effective amount of a compound capable of reducing the intracellular concentration of Aβ, such that intracellular amyloid production or accumulation is prevented or inhibited.

In another aspect, the invention relates to a method for preventing, reducing, or inhibiting amyloid production in a subject. For example, such a method comprises administering to a subject a therapeutically effective amount of a compound capable of inhibiting Aβ intracellular accumulation, such that Aβ production is prevented, reduced, or inhibited.

The invention also relates to a method for modulating amyloid-associated damage to cells, comprising the step of administering a compound capable of reducing the intracellular concentration of Aβ, such that said amyloid-associated damage to cells is modulated.

Additionally, the invention includes a method for preventing, reducing, or inhibiting amyloid production in a subject. For example, the invention includes a method which comprises administering to a subject a therapeutically effective amount of a compound capable of inhibiting β-secretase or γ-secretase, such that intracellular Aβ production is prevented, reduced, or inhibited.

The invention also includes a method for preventing cell death in a subject, the method comprising administering to a subject a therapeutically effective amount of a compound capable of preventing Aβ-mediated events that lead to cell death. In one such aspect, the method uses a compound capable of preventing Aβ-mediated events that lead to cell death such as a caspase inhibitor. For example, the caspase inhibitor may be a caspase-6 or caspase-8 inhibitor, or selected from the group consisting of pan caspase inhibitor, Z-VAD-fmk, Z-VEID-fmk, and Z-EITD-fmk.

The present invention also relates to a method for screening a potential useful compound for treating or preventing an Aβ amyloid-associated disease or condition. In this regard, the method comprises the steps of administering to a cell a compound to be screened and measuring inhibition of cell death mediated by Aβ, or measuring an intracellular. concentration of Aβ, wherein an intracellular concentration of Aβ lower than a concentration of Aβ measured for a normàl cell is indicative of said compound being useful for treating the Aβ amyloid-associated disease or condition.

The instant invention also includes a method for treating or preventing an Aβ amyloid-associated disease or condition, the method comprising the step of breaking down intracellular Aβ or causing excretion of Aβ for reducing the intracellular concentration of Aβ, thereby treating or preventing the Aβ amyloid-associated disease or condition.

Also, the invention includes a method for preventing Aβ-mediated neurotoxicity in a patient, said method comprising the step of administering to said patient an anti-apoptotic compound for inhibiting pro-apoptotic properties of Bax. The anti-apoptotic compound may be one selected from the group consisting of humanized monoclonal antibodies and polyclonal antibodies.

As used herein, “humanized” forms of non-human (e.g., rodent) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Generally, humanized antibodies are human immunoglobulins (recipient antibody) in which residues from a hypervariable region of the recipient are replaced by residues from a hypervariable region of a non-human species (donor antibody) such as mouse, rat or rabbit having the desired specificity, affinity, and capacity. In some instances, framework region (FR) residues of the human immunoglobulin are replaced by corresponding non-human residues.

Furthermore, humanized antibodies may comprise residues which are found neither in the recipient antibody nor in the donor antibody. These modifications are made to flrher refine and maximize antibody performance. In general, the humanized antibody will comprise substantially all of at least one, and typically two, variable domains, in which all or substantially all of the hypervariable loops correspond to those of a non-human immunoglobuhin and all or substantially all of the FRs are those of a human immunoglobulin sequence. The humanized antibody optionally also will comprise at least a portion of an immunoglobulin constant region (Fc), typically that of a human immunoglobulin. See, e.g.,. Jones, et al., Nature, 3221:5222 525 (1986); Reichmarm, et al., Nature, 332:323-329 (1988); and Presta, Curr. Op. Struct. Biol., 2:593-596 (1992).

The invention also pertains to a method for preventing Aβ-mediated neurotoxicity in a patient, said method comprising the step of inactivation of p53 pro-apoptotic pathway for inhibiting neurotoxicity of Aβ. In this regard, the step of inactivation may be carried out by administration to the patient of p53DN mutant which inactivates the p53 pro-apoptotic pathway.

In one preferred aspect, the invention pertains to a method for identifying a compound capable of treating an Aβ amyloid-associated disease or condition comprising assaying the ability of the compound to modulate APP nucleic acid expression or intracellular Aβ toxicity or pathological activity, thereby identifying a compound capable of treating an Aβ amyloid-associated disease or condition. In such methods of the invention, intracellular APP expression or Aβ toxicity preferably is decreased.

In another preferred embodiment, the invention pertains to a method for modulating an Aβ amyloid-associated disease or condition in a subject comprising contacting a cell of the subject with an agent that modulates intracellular APP expression or Aβ toxicity, such that an Aβ amyloid-associated disease or condition is modulated. In such methods of the invention, intracellular APP expression or Aβ toxicity preferably is decreased.

In methods of the invention, the Aβ amyloid-associated disease or condition preferably is selected from the group consisting of Alzheimer's disease, cerebral amyloid angiopathy, Down's Syndrome, or inclusion body myositis.

According to these methods of the invention, the ability of a compound to modulate intracellular APP expression or Aβ activity is determined by detecting or measuring the ability of the cell to live, i.e., cellular viability.

In other aspects, the invention includes methods for modulating cellular viability in a cell comprising contacting a cell with an APP nucleic acid expression or Aβ toxicity or pathological activity modulator, thereby modulating cellular viability in the cell.

In preferred aspected of the methods described herein, the cell is a neuronal cell, preferably a neuronal cell derived from a primate, and especially a neuronal cell derived from a human.

In one embodiment, the therapeutic agent of the invention may be an antisense or complementary Aβ peptide. An “antisense” peptide is an amino acid sequence that corresponds to that derived from a DNA sequence complementary to the normal coding sequence. The principle of antisense peptides is that the hydropathic character of a peptide derived from the coding strand will be opposite to that derived from the complementary strand. Therefore, there will be a relationship in respect of the hydropathic character, and it is expected that the antisense and sense peptides will undergo protein-protein interactions. Antisense peptides have been shown to inhibit the fibrillization and toxicity of extracellular Aβ (see, Heal, et al., Chembiochem 2002, vol. 3, pp.86-92; W002/36614, incorporated herein by reference).

In a preferred aspect, the invention relates to a method of identifying a compound capable of modulating APP nucleic acid expression or Aβ toxicity or pathological activity in a cell (preferably a neuronal cell), comprising contacting a cell with a compound; and assaying the ability of the test compound to modulate the expression of APP nucleic acid or the toxicity of Aβ, thereby identifying a compound capable of modulating APP nucleic acid -expression or Aβ toxicity or pathological activity in a cell. Such a method may frrther comprise a step of introducing AD into the cell or contacting a cell with Aβ.

Similarly, in another preferred aspect, the invention pertains to a method of identifying a compound capable of treating or preventing an amyloid-associated disease or condition, comprising contacting a cell (preferably a neuronal cell) with a compound; and assaying the ability of the test compound to modulate the expression of APP nucleic acid or the toxicity of Aβ, thereby identifying a compound capable of treating or preventing an amyloid-associated disease or condition. Such a method may further comprise a step of introducing Aβ into the cell or contacting a cell with Aβ.

In such preferred aspects, the “compound” is preferably a small molecule having a molecular weight under 2500 Daltons, peptide, antisense oligonucleotide, enzyme, antibody, ribozyme, or haptomer.

As used herein, an “introducing” step may include microinjection, contacting the cell with a liposome containing Aβ, transfection with an oligonucleotide encoding APP or Aβ, contacting the cell with a conjugate of APP gene product or Aβ with a peptide carrier, electroporation, contacting the cell with calcium chloride, contacting the cell with a DNA or RNA encoding APP or Aβ, or contacting a cell with a viral vector.

Peptide carriers may also be used to deliver peptides and proteins into cells. Small protein domains termed protein transduction domains (PTDs) can cross biological membranes efficiently and independently of transporters and specific receptors, and promote the delivery of peptides and proteins into cells (reviewed in Schwarze, et al., Trend. Pharmacol. Sci. 21:45-48, 2000).

Examples of such PTDs include TAT-derived peptides from the human, immunodeficiency virus, the third helix of the homeodomain of Antennapedia (also known as penetratin), the HSV-1 structural protein VP22, the peptide-based gene delivery system MPG, the peptide carrier Pep-1 (see, Morris, et al., Nature Biotechnology 19:1173-1176, 2001), transportan, and MAP (KLAL). Myristylated peptides can also enter cells and may be used as carriers.

Viral-mediated gene transfer can also be used for gene delivery into cells. Several classes of viral vectors are known, including but not limited to adenovirus, adeno-associated virus, herpes simplex virus, lentivirus and vaccinia virus (Larochelle, Curr Top Microbiol. Immunol. 2002, vol 261, pp.143-63). Recombinant baculovirus vectors engineered to contain mammalian cell-active promoter elements have also been used successfully for transient and stable gene delivery in mammalian cells (Kost, Trends Biotechnol. 2002, vol. 20, ppl73-80).

Such peptide and gene delivery methods may be used to administer a therapeutic agent to a subject, or to create a cell line for use in screening assays. In some instances, a transfected cell may itself be used as a vector for gene delivery. Such a cell could be an exogenous cell or, optimally, a cell isolated from a subject which has been transfected in vitro to express a compound or therapeutic agent of the invention, preferably under the control of an inducible expression system.

For the screening methods of the present invention, stable cell lines may be used. These cell lines may be, for example: immortalized cells, i.e. immortalized human or animal neurons; conditionally immortalized cells, e.g. cells in which the immortalizing agent is expressed conditionally, such that immortalization may be reversed or cells may be differentiated at the time of screening; embryonic stem (S) cells which are differentiated along a neuronal pathway, or into neurons; neuronal stem cells; or any cell line which is susceptible to Aβ toxicity. In some instances it may be advantageous to use a cell line that expresses Aβ under the control of an inducible promoter or expression system, so that Aβ expression is induced at the time of screening.

In some methods of the invention, the “assaying” step is preferably an apoptosis assay, such as one selected from the group consisting of TUNEL, measuring activation of caspases (e.g., Apo-ONE™, Homogeneous Caspase-3/7 Assay from Promega, Mafison, Wis., USA), MTT, or WST-1.

In general, apoptosis can be assayed using a wide range of assays for the quantification of cellular proliferation, viability, and cytotoxicity. Exemplary reagents and markers used in such assays for measuring cell death include the following: MTT, XTT, WST-1, lactate dehydrogenase (LDH), Alexa 568-conjugated anticoagulant, biotin-conjugated anticoagulant, fluorescein-conjugated anticoagulant, Annexin-V, activation of caspases (e.g.caspase 3), cytochrome C, cytoplasmic histone-associated DNA fragments, BrdU incorporation during DNA synthesis, [3H]-thymidine incorporation during DNA synthesis, and fluorescein-labeled cell markers (e.g.monoclonal antibodies) which bind to cytoskeletal proteins (e.g. CK18). The detection methods used in these assays may be calorimetric, photometric, radioactive or antibodγ-based (eg. ELISA, fluorescein-labeled antibodies). Propidium iodide (PI) staining can also be used as a marker of cell death. In most cases these assays and reagents are available commercially, for example, from Roche Applied Scientific, Inc., Alexis, Ambion, Biomol, Chemicon, Clontech, Genzyme Diagnostics, Immunotech, Promega, Stratagene, and Zymed.

In a preferred aspect, the screening assay is carried out against members of a combinatorial library.

In preferred methods of the invention, the peptide referred to as “Aβ” is Aβ₁₋₄₂.

Furthermore, in preferred aspects, the “Aβ amyloid-associated disease or condition” or “disease state associated with amyloidosis” is selected from the group consisting of Alzheimer's disease, cerebral amyloid angiopathy, Down's Syndrome, and inclusion body myositis.

In preferred embodiments of the screening assays described herein, the cell conditionally expresses Aβ; for example, expression of Aβ is regulated by a tetracycline-inducible gene expression system, such as the “Tet-on/off” or “Lac” system.

An inducible expression system is controlled by an external stimulus. Ideally such a system would not only mediate an “on/off” status for gene expression, but would also permit limited expression of a gene at a defined level. Some examples of sytems for controlling gene activity have been made using various inducible eukaryotic promoters, such as those responsive to heavy metal ions (Mayo, et al. (1982) Cell 29:99-108; Brinster, et al. (1982) Nature 296:39-42; Searle, et al. (1985) Mol. Cell. Biol. 5:1480-1489), heat -shock (Nouer et al. (1991) in Heat Shock Response, e.d. Nouer, L. , CRC, Boca Raton , FL, ppl67-220) or hormones, e.g. ecdysone, glucocorticoids, progesterone, or estrogen (Lee, et al. (1981) Nature 294:228-232; Hynes, et al. (1981) Proc. Natl. Acad. Sci. U.S.A. 78:2038-2042; Klock, et al. (1987) Nature 329:734-736; Israel & Kaufinan (1989) Nucl. Acids Res. 17:2589-2604).

Another strategy is to introduce regulatory elements from evolutionarily distant species such as E.coli into higher eukaryotic cells with the anticipation that effectors which modulate such regulatory circuits will be inert to eukaryotic cellular, physiology and, consequently, will not elicit pleiotropic effects in eukaryotic cells.

For example, the Lac repressor (aacR)/operator/inducer system of E.coli functions in eukaryotic cells and has been used to regulate gene expression by three different approaches: (1) prevention of transcription initiation by properly placed lac operators at promoter sites (Hu & Davidson (1987) Cell 48:555-566; Brown, et al. (1987) Cell 49:603-612; Figge, et al. (1988) Cell 52:713-722; Fuerst, et al (1989) Proc. Natl. Acad. Sci. U.S.A. 86:2549-2553: Deuschle, et al. (1989) Proc. Natl. Acad. Sci. USA. 86:5400-5405); (2) blockage of transcribing RNA polymerase II during elongation by a LacR/operator complex (Deuschle, et al. (1990) Science 248:480-483); and (3) activation of a promoter responsive to a fusion between LacR and the activation domain of herpes simples virus (ISV) virion protein 16 (VP16) (Labow, et al. (1990) Mol. Cell. Biol. 10:3343-3356; Baim, et al. (1991) Proc. Natl. Acad. Sci. U.S.A. 88:5072-5076).

In one version of the Lac system, expression of lac operator-linked sequences is constitutively activated by a LacR-VP16 fusion protein and is turned off in the presence of isopropyl-β-D-thiogalactopyranoside (IPTG) (Labow, et al. (1990), op. cit.). In another version of the system, a lacR-VP16 variant is used which binds to lac operators in the presence of IPTG, which can be enhanced by increasing the temperature of the cells (Baim, et al. (1991), op. cit.). The utility of these lac systems in eukaryotic cells may be limited, in part, because IPTG acts slowly and inefficiently in eukaryotic cells and must be used at concentrations which approach cytotoxic levels. Alternatively, use of a temperature shift to induce gene expression is likely to elicit pleiotropic effects in the cells.

Components of the tetracycline (Tc) resistance system of E. coli have also been. found to function in eukaryotic cells and have been used to regulate gene expression. For example, the Tet repressor (TetR), which binds to tet operator sequences in the absence of tetracycline and represses gene transcription, has been expressed in plant cells at sufficiently high concentrations to repress transcription from a promoter containing tet operator sequences (Gatz, C., et al. (1992) Plant J. 2:397-404).

In other studies, TetR has been fused to the activation domain of VP16 to create a tetracycline-controlled transcriptional activator (tTA) (Gossen, M. and Bujard, H. (1992) Proc. Natl. Acad. Sci. U.S.A. 89:5547-5551). The tTA fusion protein is regulated by tetracycline in the same manner as TetR, i.e., tTA binds to tet operator sequences in the absence of tetracycline but not in the presence of tetracycline. Thus, in this system, in the continuous presence of Tc, gene expression is kept off, and to induce transcription, Tc is removed.

For more examples of cell systems appropriate for use in the methods of the present invention, see, U.S. Pat. Nos. 5,589,362, 5,814,618, 6,004,941, 5,866,755, 5,912,411, 5,789,156, 5,654,168, 5,888,981, 5,859,310, 5,650,298, and 6,252,136. One such cell system is the “Tet-on/off” system from BD Bioscience Clontech (Palo Alto, Calif., U.S.A.).

The invention provides methods (also referred to herein as “screening assays”) for identifying modulators, i.e., candidate or test compounds or agents (e.g., peptides, peptidornimetics, small molecules, ribozymes, or AD antisense molecules) which bind to Aβ peptides or have an inhibitory effect on APP expression or Aβ toxicity or pathological activity. Compounds identified using the assays described herein may be useful for treating disease states associated with amyloidosis.

In another embodiment, modulators of APP expression or Aβ toxicity are identified in a method wherein a cell is contacted with a candidate compound and the expression of APP gene product or Aβ toxicity in the cell is determined. The level of expression of APP mRNA or Aβ peptide in the presence of the candidate compound is compared to the level of expression of APP MRNA or AD peptide in the absence of the candidate compound. The candidate compound can then be identified as a modulator of APP expression or Aβ toxicity based on this comparison. For example, when expression of APP MRNA or Aβ peptide is greater (statistically significantly greater) in the presence of the candidate compound than in its absence, the candidate compound is identified as a stimulator of APP expression or Aβ toxicity. Alternatively, when expression of APP mRNA or Aβ peptide is less (statistically significantly less) in the presence of the candidate compound than in its absence, the candidate compound is identified as an inhibitor of APP expression or AD toxicity.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistrγ-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic (generally under 2500 Daltons, as described further herein) and inorganic molecules (e.g., molecules obtained from combinatorial and natural product libraries). Preferred antibodies are humanized antibodies suitable for administration to humans. Also preferred are antibodies or fragments thereof that bind to Aβ.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affmity chromatography selection. The biological library approach is limited to peptide libraries, while the other four approaches are applicable to peptide, non-peptide oligomer or small molecule libraries of compounds (Lam, K. S. (1997) Anticancer. Drug Des. 12:145).

Examples of methods for the synthesis of molecular libraries can be found in the art, for example: DeWitt et al. (1993) Proc. Natl. Acad. Sci. USA 90:6909; Erb et al. (1994) Proc. Natl. Acad Sci. USA 91:11422; Zuckermann et al. (1994) J. Med Chem. 37:2678; Cho et al. (1993) Science 261:1303; Carrell et al. (1994) Angew. Chem. Int. Ed Engl. 33:2059; Carell et al (1994) Angew. Chem. Int. Ed. Engl. 33:2061; and Gallop etal. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses a Aβ peptide or biologically active portion thereof is contacted with a test compound and the ability of the test compound to modulate Aβ activity is determined. In a preferred embodiment, the biologically active portion of the Aβ peptide includes Aβ₁₋₄₂. Determining the ability of the test compound to modulate Aβ activity can be accomplished by monitoring, for example, apoptosis or cell viability. The cell, for example, can be of mammalian origin, e.g., a human neuron cell.

This invention further pertains to novel agents identified by the above-described screening assays. Accordingly, it is within the scope of this invention to further use an agent identified as described herein in an appropriate animal model. For example, an agent identified as described herein (e.g., an Aβ modulating agent, an, antisense Aβ nucleic acid molecule, an Aβ-specific antibody, or an Aβ-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Additionally, gene expression patterns may be utilized to assess the ability of a compound. to ameliorate (intracellular) Aβ-associated disease symptoms. For example, the expression pattern of one or more genes may form part of a “gene expression profile” or “transcriptional profile” which may be then be used in such an assessment. “Gene expression profile” or “tanscriptional profile,” as used herein, includes the pattern of MRNA expression obtained for a given tissue or cell type under a given set of conditions. Such conditions may include, but are not limited to, transcription, translation, or expression of Aβ or APP.

Gene expression profiles may be generated, for example, by utilizing a differential display procedure, Northern analysis and/or RT-PCR. In one embodiment, Aβ gene sequences may be used as probes and/or PCR primers for the generation and corroboration of such gene expression profiles. Gene expression profiles may also be generated using DNA array technology.

The present invention provides for both prophylactic and therapeutic methods of treating a subject, e.g., a human, at risk of (or susceptible to) a disease state associated with amyloidosis such as Alzheimer's disease, Down's Syndrome, cerebral amyloid angiopathy, or inclusion body myositis.

As used herein, “treatment” of a subject includes the application or administration of a therapeutic agent to a subject, or application or administration of a therapeutic agent to a cell or tissue from a subject, who has a diseases or disorder, has a symptom of a disease or disorder, or is at risk of (or susceptible to) a disease or disorder, with the purpose of curing, healing, alleviating, relieving, altering, remedying, ameliorating, improving, or affecting the disease or disorder, the symptom of the disease or disorder, or the risk of (or susceptibility to) the disease or disorder.

As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics.

“Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype,” or “drug response genotype”).

Thus, another aspect of the invention provides methods for tailoring a subject's prophylactic or therapeutic treatment with either the Aβ modulators of the present invention or Aβ modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for preventing in a subject, a disease state associated with amyloidosis by administering to the subject an agent which modulates APP expression or Aβ toxicity or pathological activity, e.g., modulation of apoptosis or viability upon exposure to Aβ in, e.g., a neuron.

Subjects at risk for a disease state associated with arnyloidosis can be identified by, for example, any or a combination of the diagnostic or prognostic assays described herein. Administration of a prophylactic agent can occur prior to the manifestation of symptoms characteristic of aberrant APP expression or activity, such that a disease state associated with amyloidosis is prevented or, alternatively, delayed in its progression. Depending on the type of Aβ aberrancy, for example, an Aβ molecule, Aβ agonist or Aβ antagonist agent can be used for treating the subject. The appropriate agent can be determined based on screening assays described herein.

Another aspect of the invention pertains to methods for treating a subject suffering from a disease state associated with amyloidosis. These methods involve administering to a subject an agent which modulates APP expression or activity (e.g., an agent identified by a screening assay described herein), or a combination of such agents. In another embodiment, the method involves administering to a subject a Aβ-modulating compound or nucleic acid molecule (e.g., antisense) as therapy to compensate for aberrant or unwanted APP expression or Aβ toxicity or pathological activity.

Inhibition of Aβ activity is desirable in situations in which Aβ is abnormally upregulated and/or in which decreased Aβ activity is likely to have a beneficial effect, e.g., an Aβ amyloid-associated disease or condition, thereby ameliorating a disease state associated with amyloidosis such as Alzheimer's disease, Down's Syndrome, cerebral amyloid angiopathy, or inclusion body myositis in a subject.

The agents which modulate Aβ activity can be administered to a subject using pharmaceutical compositions suitable for such administration. Such compositions typically comprise the agent (e.g., small molecule, nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution (e.g. Ringer's solution), fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates Aβ activity in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fasidic acid derivatives. Transmucosal administration can be accomplished through the use of nasal sprays or suppositories. For transdermal administration, the active compounds are formulated into ointments, salves, gels, or creams as generally known in the art.

The agents that modulate Aβ activity can also be prepared in the form of suppositories (e.g., with conventional suppository bases such as cocoa butter and other glycerides) or retention enemas for rectal delivery.

In one embodiment, the agents that modulate Aβ activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates Aβ activity and the particular therapeutic effect to be achieved, and the limitations inherent in the art of compounding such an agent for the treatment of subjects.

Many drug candidate compounds identified by the methods of the invention may be sufficiently lipophilic to cross the blood brain barrier (“BBB”), or they may enter the brain by an active transport mechanism. Still other drug candidates may be formulated to enhance BBB penetration using methods known in the art and as described herein. For example, compounds of the invention can be formulated in liposomes. For methods of manufacturing liposomes, see, e.g. U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., V. V. Ranade (1989) J. Clin. Pharmacol. 29:685). Exemplary targeting moieties include folate, biotin, mannosides, antibodies, surfactant protein A receptor and gpl20. In a preferred embodiment, the therapeutic compounds of the invention are formulated in liposomes; in a more preferred embodiment, the liposomes include a targeting moiety.

A means of ensuring that a compound of the invention crosses the BBB is to couple it to a BBB transport vector (for review of BBB transport vectors and mechanisms, see, Bickel, et al., Adv. Drug Delivery Reviews, 46:247-279, 2001). Exemplary transport vectors include cationized albumin or the OX26 monoclonal antibody to the transferrin receptor; these proteins undergo absorptive-mediated and receptor-mediated transcytosis through the BBB, respectively. Examples of other BBB transport vectors that target receptor-mediated transport systems into the brain include factors such as insulin, insulin-like growth factors (IGF-I, IGF-II), angiotensin II, atrial and brain natriuretic peptide (ANP, BNP), interleukin I (IL-1) and transferrin. Monoclonal antibodies to the receptors which bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties, e.g., glucose, monocarboxylic acids, e.g., lactic acid, neutral amino acids, e.g., phenylalanine, amines, e.g., choline, basic amino acids, e.g., arginine, nucleosides, e.g., adenosine, purine bases, e.g., adenine, and thyroid hormone, e.g., triiodothyridine. Antibodies to the extracellular domain of nutrient transporters can also be used as transport vectors. Other possible vectors include angiotensin II and ANP, which may be involved in regulating BBB permeability.

In some cases the bond linking the therapeutic compound to the transport vector may be cleaved following transport into the brain in order to liberate the biologically active compound. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself is a potential drug transport vector.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such Aβ modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays.

A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of Aβ-modulating agent (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an Aβ-modulating agent can include a single treatment or, preferably, can include a series of treatments.

In a preferred example, a subject is treated with an Aβ-modulating agent in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of Aβ-modulating agent used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, substituted hydrocarbons, vitamins, amino acids (including unnatural amino acids), sterols, terpenes, polyketides and polyacetates, lipids, alkyloids, fatty acids derivatives, peptides, peptidomimetics, amino acids, amino acid analogs, polynucleotides, polynucleotide analogs, nucleotides, nucleotide analogs, and organic or inorganic compounds (i.e., including heteroorganic and organometallic compounds) having a molecular weight less thin about 10,000 grams per mole, organic or inorganic compounds having a molecular weight less than about 5,000 grams per mole, organic or inorganic compounds having a molecular weight less than about 2,500 grams per mole, organic or inorganic compounds having a molecular weight less than about 1,000 grams per mole, organic or inorganic compounds having a molecular weight less than about 500 grams per mole, and pharmaceutically acceptable salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethylsulfonate, and laurylsulphonate salts and the like. (see, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 microgranis per kilogram).

It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The present invention may be more readily understood by referring to the following examples, which are given to illustrate the invention rather than to limit its scope.

### EXAMPLES

Considerable attention in the art has been devoted to the toxicity of extracellular amyloid β peptides in Alzheimer's Disease. However, extracellular Aβ, even at high concentrations, does not induce cell death in primary human neuron cultures, in most transgenic animal models with extensive extracellular Aβ deposits, and weakly correlates with AD. In the present invention, it was found that intracellular forms of Aβ₁₋₄₂ are toxic to neurons.

Microinjection of Aβ₁₋₄₂ peptide or cytosolic Aβ₁₋₄₂ cDNA-expressing constructs rapidly induces cell death of primary human neurons. In contrast, Aβ₁₋₄₀, Aβ₄₀₋₁, or Aβ₄₂₋₁ peptides, cytosolic Aβ₁₋₄₀ or secreted Aβ₁₋₄₂ and Aβ₁₋₄₀ cDNA expressing constructs were not observed to be toxic. As little as 1 pM concentration or 1500 molecules of Aβ₁₋₄₂ peptides is neurotoxic and non-fibrillized peptides are as neurotoxic as fibrillized Aβ₁₋₄₂ peptides. In contrast, Aβ₁₋₄₂ peptides were not observed to be toxic to human primary astrocytes, human neuroblastoma La—N-1 and M17, human teratocarcinoma NT2, rat NIH 3T3 fibroblasts, or baby hamster kidney (BHK) cell lines. 5 It is further demonstrated herein that inhibition of de novo protein synthesis and caspases protects against Aβ toxicity. In addition, Bcl-2, Bax neutralizing antibodies, and p53R273H dominant negative mutant completely eliminate Aβ₁₋₄₂-mediated human neuronal cell death. Talcen together, the data reported herein directly demonstrate that intracellular Aβ₁₋₄₂ is selectively cytotoxic to human neurons and mediated at least in part by the p53-Bax cell death pathway.

To determine if intracellular cytoplasmic Aβ may be detrimental to neurons, Aβ₁₋₄₀, Aβ₁₋₄₂, and control reverse peptide Aβ₄₀₋₁ were microinjected in primary cultures of human neurons. It was found that Ap,₄₂ is selectively extremely toxic to human neurons and regulates cell death by activation of the p53 and Bax pro-apoptotic pathway.

In accordance with the present invention, there is provided a new method for screening for drugs against AD and for treating or preventing AD by administering a compound that prevents intracellular accumulation of, or reduces the intracellular concentration of Aβ₁₋₄₂. To directly address the intracellular toxicity of Aβ₁₋₄₀ and Aβ₁₋₄₂, human neurons in primary culture were microinjected with these peptides using the reverse A40-1 peptide as control. It was found that 25×10⁻²¹ moles of Aβ₁₋₄₂ intracellular are sufficient to induce apoptosis in 70% of the microinjected neurons within 24 hours after injection and 90-95% apoptosis 96 hours after injection.

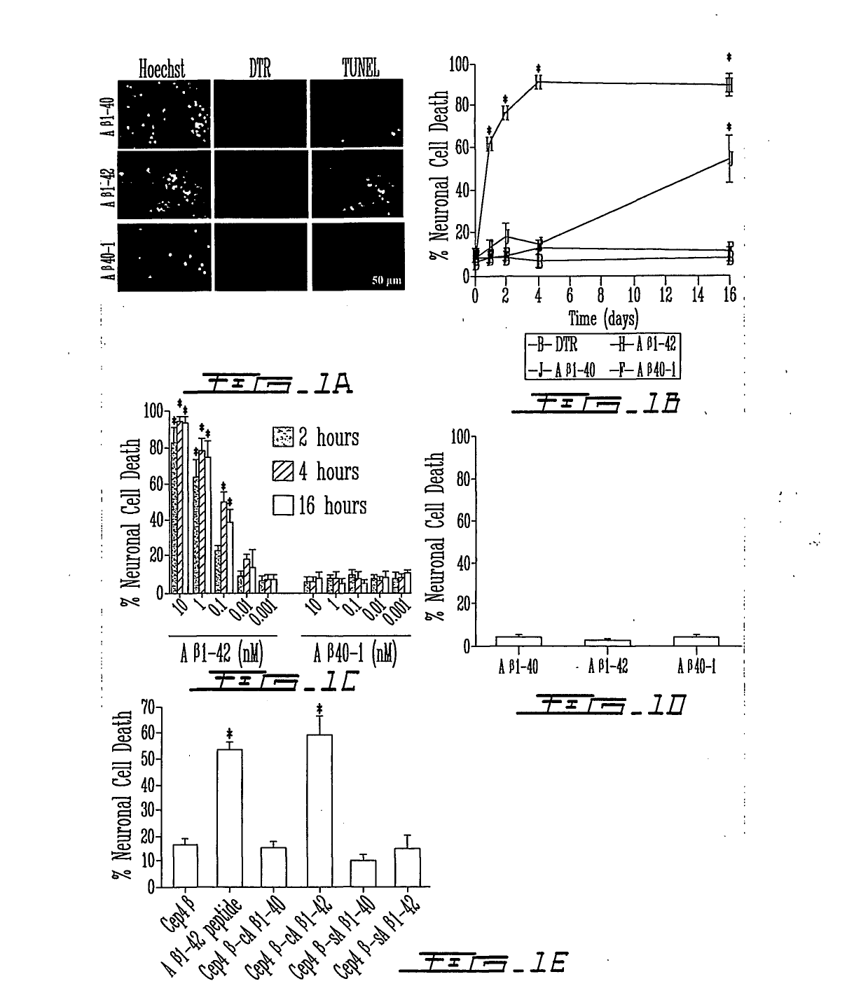

The Aβ₄₀₋₁ control peptide was found to be not toxic and the Aβ₁₋₄₀ displays significant apoptosis only 16 days after microinjection. Assuming the volume of a neuron is 5 nL, this injection is equivalent to 50 pM concentration in the cytosol of the neuron (25×10⁻²¹ moles/cells, i.e. in 5 niL, represents 5×10⁻¹¹ moles/Litre or 50 pmoles/Litre). Furthermore, the concentration of Aβ₁₋₄₂ can be reduced 100 to 1000 fold and still exert significant toxicity in these neurons (FIG. 1C).

In FIG. 1C, the neurons were microinjected with decreasing concentrations of the Aβ42 and control Aβ₄₀₋₁ peptides. The Aβ42 is toxic with as few as 25×10⁻²³ moles (150.5 molecules) while the Aβ₄₀₋₁ remains non-toxic. At best, Aβ42 is known in the art to be toxic at concentration of about 10-100 μM, as this concentration has been determined in a medium or extracellularly.

These results illustrate two issues regarding Aβ toxicity: The first is that intracellular Aβ42 is a specifically toxic amyloid peptide in the cytosol of human neurons compared to Aβ₁₋₄₀. The second is that very low physiological concentrations are sufficient to induce neuronal cell death.

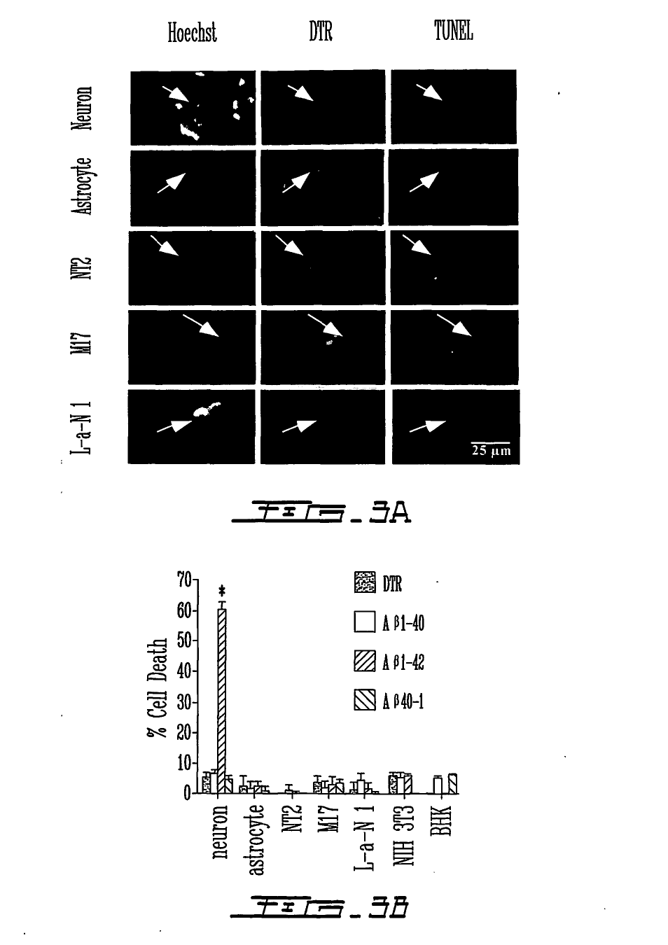

To address if neurons are selectively vulnerable to Aβ42 toxicity, microinjections in primary cultures of human astrocytes, teratocarcinoma NT2, neuroblastoma M17, and LaN-1 cell lines were repeated. In contrast to the human neurons, these cells completely resist Aβ₁₋₄₂ toxicity even with a ten times higher dose (FIG. 3B).

In FIG. 3B, human primary astrocytes, teratocarcinoma NT2, neuroblastoma M17 and LaN-1 cell lines were injected with 8×10⁻²⁰ moles (48184 molecules) and 8×10⁻¹⁹ moles (481840 molecules) of Aβ42 and incubated for twenty-four hours. While neurons die within twenty-four hours with these doses of Aβ42, the other various cell lines remain resistant to the Aβ42. The volumes of the intracellular compartment of astrocytes, M17, NT2, and LaN-1 cells are respectively of 30 nL, 35 nL, 66 nL, and 57 nL. Table 1 illustrates the corresponding quantity, volume and concentration injected in astrocytes.

TABLE 1

| Injected moles | Volume | Concentration |
|---|---|---|
| 8 × 10⁻²⁰ | 30 nL | 2.67 pM |
| 8 × 10⁻¹⁹ | 30 nL | 26.7 pM |

In the human neurons tested, the intracellular Aβ exits the neurons rapidly, whereas the extracellular form of Aβ accumulates in the culture medium. These results show that the rapid secretion of Aβ is a detoxifying mechanism designed to avoid accumulation of intracellular amyloid β peptide.

Intracellular Aβ₁₋₄₂ toxicity may precede extracellular amyloid deposition and other pathological features of AD (D'Andrea, M. R., Nagele, R. G., Wang, H. Y., Peterson, P. A. & Lee, D. H., Histopathology 38, 120-34 (2001)). Studies show that accumulation of intracellular Aβ₁₋₄₂ occurs in transgenic mice harboring the V717F APP mutation (Li, Q. X., Maynard, C., Cappai, R., McLean, C. A., Cherny, R. A., et al., J. Neurochem. 72, 2479-87 (1999)) and these mice show a decrease in presynaptic terminals and neurons before the deposition of extracellular Aβ (Masliah, E., Sisk, A., Mallory, M., Mucke, L., Schenk, D. .& Games, D., J. Neurosci. 16, 5795-811 (1996); and Hsia, A. Y., Masliah, E., McConlogue, L., Yu, G. Q., Tatsuno, G., et al., Proc. Natl. Acad. Sci. USA. 96, 3228-33 (1999). Presenilin I mutations L286V, H163R show neuronal accumulation of Aβ₁₋₄₂ and accelerated neurodegeneration in absence of amyloid plaque formation (Chui, D. H., Tanahashi, H., Ozawa, K., Ikeda, S., Checler, F., et al., Nat. Med. 5, 560-4 (1999)).

cDNA Clones. Human Bcl-2 cDNA, Bax α cDNA, and APP695 cDNA were cloned into pCep4β (Invitrogen Canada, Burlington, ON, Canada). The p53 wild type and p53R273H dominant negative cDNA may be cloned in the pCMV-NEO vector. These cDNAs were purified through GlassMAXTM (Gibco-BRL, Rockville, Mass.) and diluted at 30 ng/μl in PBS before injection.

Primers were designed to amplify secreted and cytosolic Aβ40 (the sequence of which is DAEFRHDSGYEVHHQKLVFFAEDVGSNKGAIIGLMVGGVV (SEQ ID NO: 1)and Aβ42 (the sequence of which is DAEFRHDSGYEVHHQKLVFFAEDVGSNKGAIIG LMVGGVVIA (SEQ ID NO: 2) from APP695. These primers amplify the entire Aβ sequence and an additional methionine ATG codon was added at the 5′ end and a stop codon at the 3′ end, to ensure translation.

For amplification of cytosolic Aβ, primers are as follows: Aβ40/42 forward primer is 5′-TCACTCGAGAATGGATGCAGAATTCC GACAT-3′ (SEQ ID NO:3) and contains a built-in 5′ XhoI site, Aβ42 reverse primer is 5′-ATGGATCCTTACGCTATGACA ACACCGAA-3′ (SEQ ID NO:4) and has a 3′ BamHI site, Aβ40 reverse primer is 5′-TCGATCCTTAGACAACACCGCCCACCATG-3′ (SEQ ID NO:5) and has a 3′ BamHI site.

For amplification of secreted Aβ, the signal peptide (SP) sequence of APP was amplified with Aβ-SP1 forward primer: 5′-TTACTCGAGATGCTGC CCGGTTTGGCA-3′ (SEQ ID NO:6) containing a XhoI site and Aβ-SP2 reverse primer: 5′-GGAATTCTGCA TCCATCGCCCGAGCCGTCCAGGC-3′ (SEQ ID NO:7) which contains a 3′ EcoRI site. A ligation between the EcoRI cleaved PCR amplified Aβ coding sequence and the EcoRI cleaved signal peptide sequence was re-amplified with the Aβ40/42 forward primer and Aβ40 or Aβ42 reverse primers.

The PCR amplified signal peptide and Aβ sequences were cloned into the pBSKII prokaryotic and Cep4p eukaryotic episomal vectors through the XhoI/BamHI restriction sites. All clones were restriction mapped and sequence to confirm the sequence.

Neutralizing Bax antibodies. Monoclonal anti-Bax 6A7 (amino acids 12-24: Pharmingen, San Diego, Calif.) and 2D2 (amino acids 3-16: Trevigen, Gaithersburg, Md.), polyclonal anti-Bax N-20 (Santa Cruz Biotechnology, Santa Cruz, Calif.), monoclonal anti-APP 22C11 (Roche Molecular Biochemicals, Laval, PQ, Canada), mouse IgG or rabbit sera were diluted at 50 μg/ml (for 6A7, 2D2, mouse IgG and 22C11) or 25 μg/ml (for polyclonal anti-Bax and rabbit sera) in PBS before use. A toxicity curve was done to determine this concentration as the highest non-toxic concentration that can be injected in neurons.

Cell cultures. Primary cultures of human neurons and astrocytes were prepared as described in LeBlanc, A., J. Neurosci. 15:7837-46 (1995), incorporated herein by reference. Fresh brain tissues were dissociated with 0.25% trypsin (Gibco-BRL) in phosphate buffered saline (PBS) at 37° C. for 15 minutes. The trypsin was inactivated. by adding 10% decomplemented fetal bovine serum (FBS, HyClone, Logan, Utah.) to the mixture. The dissociated tissues were triturated in 0.1 mg/ml DNaseI (Gibco-BRL) to obtain a homogeneous mixture. The mixture was filtered successively through 130 and 70 gm filters and centrifuged at 15,000 g for 10 minutes at 10° C. to pellet the cells. The cell pellet was washed once with PBS and once with minimal essential media (MEM, Gibco-BRL) in Earle's balanced salt solution containing 0.225% sodium bicarbonate, 1 mM sodium pyruvate, 2 mM L-glutamine, 0.1% dextrose, 1×antibiotic Pen-Strep (all from Gibco-BRL) and 5% decomplemented FBS.

The cells were plated on poly-L-lysine-coated ACLARTM (Cat. No.: 33C; thickness: 0.5 mm; Allied Chemical, Minneapolis, Minn.) coverslips at a density of 3×10⁶ cells/ml. Neurons and astrocytes were incubated at 37° C. in the MEM media with 5% CO₂, and the media were changed every 48 hours. For neuron culture, 1 mM fluorodeoxyuridine (FDU, Gibco-BRL) was applied for the first 3 feedings and after that, used weekly to prevent proliferation of dividing cells. In general, the neurons attach to the coverslips within 24 hours and develop dense neuritic networks within 3 days. Typically, the cultures contain 90-95% neurons and 5-10% astrocytes. Microinjection or treatment was performed 10 days after plating for neurons and astrocytes.

Human neuroblastoma M17 cells were cultured on ACLAR™ coverslips at 1×10⁶ cells/ml in OPTI-MEM (Gibco-BRL) containing 5% FBS. Microinjection was performed when the cells reached 70-80% confluence on the coverslips. Human teratocarcinoma NT2 (Stratagene, La Jolla, Calif.) and neuroblastoma La-N-1 cells were cultured on ACLAR™ coverslips at 1×10⁶ cells/ml in DMEM (Gibco-BRL) containing 10% FBS. Microinjection was performed when the cells were 50% confluent.

Aβ peptides. Aβ peptides (Bachem, King of Prussia, Pa.) were dissolved in sterile distilled water at 25 μM and incubated at 37° C. for 5 days. The peptides stock solutions were frozen and diluted in 1×PBS immediately before microinjection. For the preparation of non-fibrillar and fibrillar Aβ peptides, a 25 μM solution of disaggregated Aβ₁₋₄₀ and Aβ₁₋₄₂ peptides (American Peptide Co. CA) was prepared in 5 mM Tris buffer pH 7.4. A 250 μl aliquot was diluted to 0.25 μM and immediately frozen at −20° C. in aliquots of 50 μl. The remaining solution was incubated at 37° C. in Eppendorf tubes with continuous mixing by inversion. After incubation, the samples were removed, vortexed, sonicated twice for 1 minute in a bath type sonicator (ELMA GmbH & Co. KG, Germany) and frozen at −20° C. in 5041 aliquots. Each aliquot was used once to avoid possible effects of freeze and thaw cycles.

Electron Microscopy. A 3 μl aliquot of Aβ peptide was placed on freshly cleaved mica plates (BioForce Laboratory Inc., Calif.). The specimens were air dried and subsequently transferred to a Balzers High-Vacuum Freeze-Etch Unit (model 301). Under a 1.3×10⁻⁴ Pa vacuum, the specimens were shadowed with platinum (BAL-TEC EM-Technology and Application, N.H.) at a 30° angle and coated with a carbon film platinum (BAL-TEC EM-Technology and Application, N.H.). The replicas were detached from the mica by flotation using deionized water and transferred onto a 300-mesh grid (Canemco Inc., St Laurent, QC). The grids were examined using a Joel 200FX transmission electron microscope (Joel, USA) at 20,000× magnification.

Microinjection. Thin-walled Borosilicate glass capillaries (OD 1.0 mm, ID 0.5 mm) with microfilament (MTW1OOF-4, World Precision Instrument, Sarasota, Fla.) were pulled with a Flaming/Brown Micropipefte Puller (β-87, Sutter, Novato, Calif.) to obtain injection needles with a tip diameter of ˜0.5 μm. Microinjection was performed in the cytosol of each cell using the Eppendorf Microinjector 5246 (Hamburg, Germany) and Burleigh Micromanipulator MIS-5000 (Victor, N.Y.). Human neurons were injected with 25 pl/shot at an injection pressure of 100 hPa, a compensation pressure of 50 hPa, and an injection time of 0.1 s. Human astrocytes, M17, NT2 and La—N-1 cells were injected with 8 pl/shot at an injection pressure of 50 hPa, a compensation pressure of 30 hPa and an injection time of 0.1 s.

The diluted peptides were injected at the indicated concentrations with 100 μg/ml dextran Texas Red (DTR) (MW: 3000, Molecular Probes, Eugene, Oreg.) as a fluorescent marker to recognize the injected cells. Approximately 90% neurons and NT2 cells, and 50% astrocytes, M17 and La-N-1 cells are retained on the coverslips for at least 16 days after injection as indicated by DTR staining.

Measurement of neuronal apoptosis. Cells were fixed in freshly prepared 4% paraformaldehyde/4% sucrose for 20 minutes at room temperature and permeabilized in 0.1% Triton X-100, 0.1% sodium citrate on ice for 2 minutes. Terminal deoxynucleotidyl transferase-mediated dUTP nick end labeling (TUNEL) was performed using the In Situ Cell Death Detection Kit I as described by the manufacturer (Roche Molecular Biochemicals). The percentage of cell death was determined as the ratio of the number of DTR-TLUNEL-double-positive cells over the total number of DIR-positive cells.

Hoechst staining was used to recognize cell nuclei and detect apoptotic nuclear condensation and fragmentation. Hoechst dye (Intergen, Purchase, N.Y.) was dissolved in sterile distilled water at 200 fglml and diluted 500 times in PBS immediately before staining. After the incubation for TUNEL staining, cells were washed 3 times for 10 minutes each in PBS. The 200 μg/ml of diluted Hoechst dye was added onto the coverslips to cover the cells. Cells were incubated at room temperature in the dark for 15 minutes, washed 3 times for 10 minutes each in PBS, washed once in water for 5 minutes, and mounted with Immunon™ mounting medium (Shandon, Pittsburgh, Pa.) onto glass slides to be observed under the fluorescence microscope.

Treatment with caspase inhibitors, cycloheximide, actinomycin D. Caspase pan inhibitor, Z-Valine-Alanine-Aspartic acid-fluoromethylketone (Z-VAD-fmk) (Biomol, Plymounth Meeting, PA); caspase-6 inhibitor, Z-Valine-Glutamic acid-Isoleucine-Aspartic acid-fmk (Z-VEID-fnk); caspase-3 inhibitor, Z-Aspartic acid-Glutamic acid-Valine-Aspartic acid-fmk (Z-DEVD-fmk); and caspase-8 inhibitor, Z-Isoleucine-Glutamic acid-Threomine-Aspartic acid-fmk (Z-IETD-fmk) (Sigma, Oakville, ON, Canada) were dissolved in 100% dimethylsulphoxide (DMSO, Sigma) at 5 mM and were diluted at 5 μM into culture medium immediately before use. The medium was changed every 48 hours.

Stock solutions of 5 mg/ml cycloheximide and 200 IM actinomycin D were made in sterile distilled water and diluted to 5 μg/ml for cycloheximide and 5 μM for actinomycin D in the culture medium immediately before use.

Statistical evaluation. The statistical significance of difference between treatments was determined by Statview 5.01 using one-way or two-way ANOVAs with post hoc tests. The Dunnett's test was used when comparing multiple groups to a certain group. The Sheffé's test was applied when comparing between groups. A p value less than 0.05 was taken as the criteria for statistical significance.

Intracellular Aβ₁₋₄₂, but not Aβ₁₋₄₀ or Aβ₄₀₋₁ is neurotoxic to human neurons. To directly determine if intracellular Aβ is toxic to human neurons, aged Aβ₁₋₄₀, Aβ₁₋₄₂ or the reverse control peptide Aβ₄₀₋₁ were microinjected into the cytoplasm of primary cultured human neurons (FIG. 1A).

FIG. 1A represents fluorescent photomicrographs of microinjected neurons. Neurons were microinjected (DTR) with the peptides and incubated 24 hours before staining for TUNEL for cell death or Hoechst for nuclear stain.

Aβ₁₋₄₂ induces significant cell death in 60% of microinjected neurons within 24 hours after injection. Cell death flirther increases to 78 and 90% at 48 and 96 hours after injection. However, Aβ₁₋₄₀, the control reverse peptide Aβ₄₀₋₁, or the fluorescent marker dye, dextran Texas Red (DTR) do not affect cell viability between 24 and 96 hours (FIG. 1B).

In FIG. 1B, aged Aβ₁₋₄₀, Aβ₁₋₄₂, and Aβ₄₀₋₁ peptides (10 nM) were microinjected into the cytosol of human neurons and cell death was measured by TUNEL at 1, 2, 4 and 16 days after injection. The data represent the mean + SEM of 3 independent experiments.

Two-way ANOVAs (dfₜᵢₘₑ=4; dfₜᵣₑₐₜₘₑₙₜ=3) followed by Sheffé's test were performed to determine the statistical significance between Aβ-injected and control DTR-injected neurons. The symbol “*” represents a probability of p<0.01. Sixteen days after injection, Aβ₁₋₄₀ induces 50% cell death.

Since 25 pl/shot of a 10 nM concentration of peptide were injected, the above results indicate that 0.25×10⁻¹⁸ moles of Aβ₁₋₄₂ delivered into the neuron cytosol is sufficient to induce rapid neuronal death. A 100-fold dilution in the amount injected still induces 50% cell death in these neurons (FIG. 1C).

In FIG. 1C, various doses of Aβ₁₋₄₂ and Aβ₄₀₋₁ were injected into human neurons and cell death was determined by TUNEL staining at 2, 4 or 16 days after injection. The data represent the mean + SEM of 3 independent experiments. Two-way ANOVAs (dfₜᵢₘₑ=2; dfₜᵣₐₜₘₑₙₜ=29) followed by Sheffé's test were performed to determine the statistical significance. The symbol “*” represents a probability of p<0.01.

Using confocal microscopy, the volume of these human neurons was estimated at 4.97 nL (n=100). The nuclei occupy over 50% of the cell, so the cytosolic area is around 2.5 nL. Therefore, the actual toxic concentration of injected Aβ₁₋₄₂ is 0.25×10⁻¹⁸ to 0.25×10⁻²⁰ moles/2.5 nL, which equals 1×10⁻¹⁰ to 1×10⁻¹² M, or 1 to 100 pM. These neurons do not undergo cell death even with 10 μM of extracellular Aβ₁₋₄₂, Aβ₁₋₄₀ or Aβ₄₀₋₁, a concentration known to induce cell death in a variety of neuronal cell lines (Klein, W. L., et al., Trends Neurosci. 24:219-24, 2001; and Paradis, E., et al., J. Neurosci. 16:7533-7539, 1996).

To test if this particular batch of peptide might be neurotoxic, neurons were treated with 10 μM of peptides for 24 hours. Neither extracellular Aβ₁₋₄₀, Aβ₁₋₄₂, or Aβ₄₀₋₁ are toxic to these neurons (FIG. 1D).

In FIG. 1D, human neurons were exposed to 10μM extracellular Aβ₁₋₄₀o, Aβ₁₋₄₂ and Aβ₄₀₋₁ for 24 hours and stained with propidium iodide to reveal cellular nuclei and TUNEL to reveal cell death. Therefore, the toxicity of intracellular Aβ₁₋₄₂ is at least 100,000 times greater than extracellular Aβ.

These results indicate that an infinitesimal amount of intracellular Aβ₁₋₄₂ is detrimental to human neurons. Calculation of the number of molecules of Aβ₁₋₄₂ injected in neurons based on the Avogadro number shows maximal toxicity with 150,055 molecules and 50% toxicity with 1505.5 molecules/neuron.

Finally, to confirm the toxicity of naturally produced intracellular Aβ peptides, neurons were microinjected with cDNA constructs expressing cytosolic or secreted Aβ₁₋₄₀ and Aβ₁₋₄₂. The expression of the Aβ peptide was verified by coupled in vitro transcription/translation and transiently transfected M17 cells.

Cep4β episomal cDNA constructs were made to express cytosolic Aβ₁₋₄₀ and Aβ₁₋₄₂ (cAβ) or secreted Aβ₁₋₄₀ and Aβ₁₋₄₂ (sAβ). The expression of Aβ peptides with and without signal peptide is shown in the in vitro translated proteins in absence or presence of microsomes. Immunoprecipitated Aβ peptides from the media or cellular lysate of M17 transiently transfected cells.

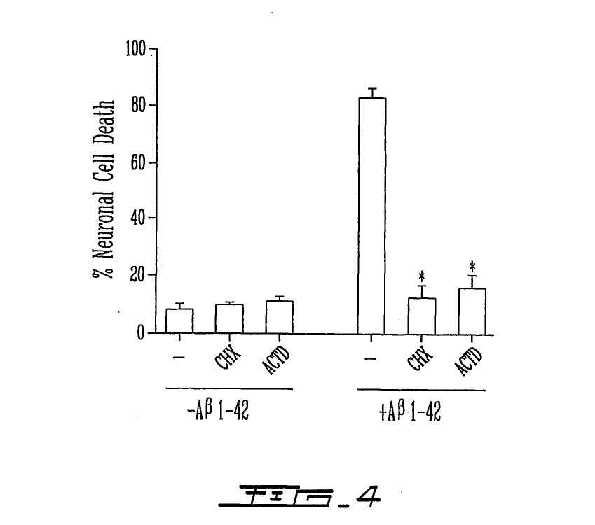

FIG. 4E represents the neuronal cell death obtained when microinjecting these cDNA constructs in the neurons instead of the peptides. As observed with the synthetic Aβ₁₋₄₂ peptide, only the cytosolically expressed Aβ₁₋₄₂ was toxic, whereas secreted Aβ₁₋₄₂ or cytosolic or secreted Aβ₁₋₄₀ did not induce cell death in neurons.

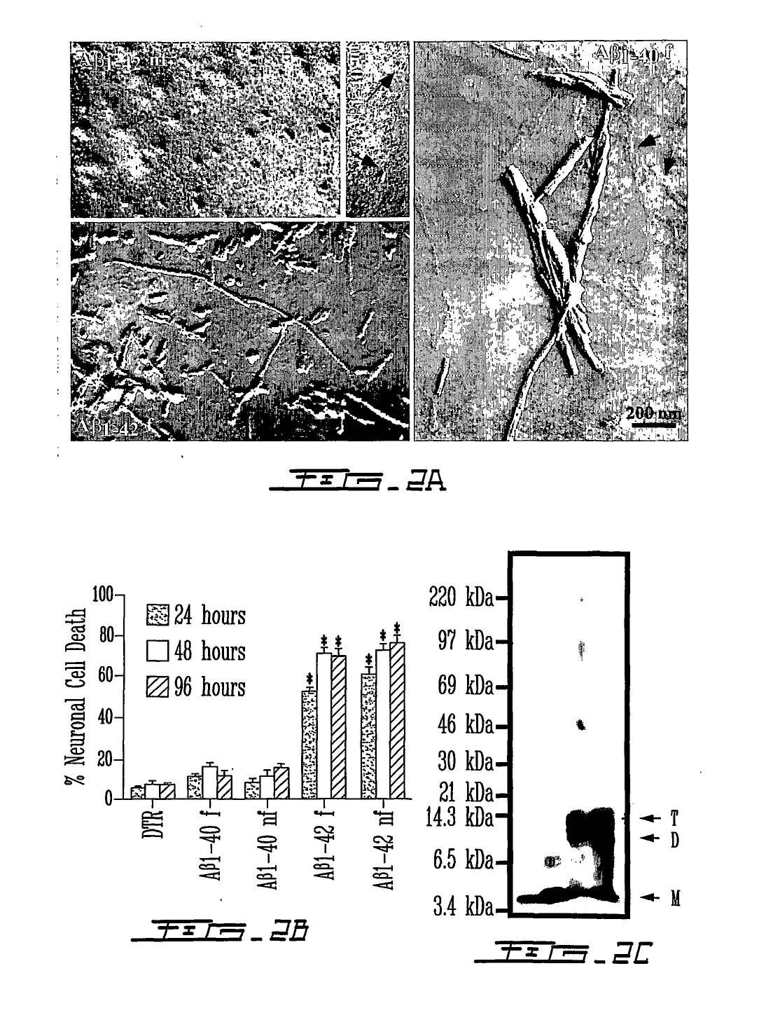

Non-fibrillized Aβ₁₋₄₂ is neurotoxic. Because the fibrillar form of Aβ is commonly seen in the senile plaques in AD brains and is proposed to be more toxic than soluble Aβ (Pike, C. J., et al., Journal of Neuroscience. 13:1676-1687, 1993), the toxicity of both fibrillized, and non-fibrillized Aβ peptides was examined. Transmission electron microscopy on the preparation of Aβ confirms the fibrillar and non-fibrillar nature of the Aβ preparation (FIG. 2A).

FIG. 2A illustrates electron micrographs of non-fibrillized (nf) and fibrillized (f) Aβ₁₋₄₀ and Aβ₁₋₄₂. The non-fibrillized Aβ₁₋₄₂ peptides show well defined globular as well as diffluse aggregate morphology. Similar well defined globular structures are also seen in the non-fibrillized Aβ₁₋₄₀ preparation. These structures are much less abundant in the Aβ₁₋₄₀ than in the Aβ₁₋₄₂ preparation. The fibrillized Aβ₁₋₄₂ peptide shows a heterogeneous mixture of fibrils of various sizes, protofibrils and globular structures. The fibrils in the Aβ ₁₋₄₀ preparation were less heterogeneous and consisted of thick fibrillar aggregates and thin aligned fibrils. The large Aβ₁₋₄₀ fibrils do not appear to pass through the micropipet and thus were unlikely to have been injected in neurons. However, some of the fibrils from the Aβ₁₋₄₂ preparation did pass through the pipet. Both fibrillized and non-fibrillized preparations of Aβ₁₋₄₂ induce 50-90% neuronal cell death between 24-96 hours after injection (FIG. 2B).

In FIG. 2B, soluble or fibrillar Aβ₁₋₄₀ or Aβ₁₋₄₂ (10 nM) were injected into human neurons and neuronal cell death assessed by TUNEL staining at 24, 48 and 96 hours after injection. The data represent the mean + SEM of 3 independent experiments. One-way ANOVA (df=14) followed by Sheffé's test were performed to determine the statistical significance between Aβ-injected and control DTR-injected neurons. The symbol “*” represents a probability of p<0.01. In contrast, neither fibrillized nor non-fibrillized Aβ₁₋₄₀ cause significant cell death. Previously, it was shown that dimers/oligomers of Aβ are toxic to neurons when applied in the extracellular niillieu (Walsh, D. M., et al., J. Biol. Chem. 274:25945-52, 1999).

Western blot analysis of the non-fibrillized and fibrillized Aβ peptides shows that the Aβ₁₋₄₂ peptide forms aggregates with the expected size of Aβ dimers, trimers and oligomers. In contrast, the non-fibrillized and fibrillized Aβ₁₋₄₀ peptides show a 6.5 kDa aggregate and with long exposure a smear of higher MW oligomers. Taken together, these data show that the dimers and trimers of the non-fibrillized and fibrillized intracellular Aβ₁₋₄₂ peptides are the toxic form of the intracellular Aβ peptides.

FIG. 2C represents western blot analysis of fibrillized or non-fibrillized Aβ₁₋₄₀ and Aβ₁₋₄₂ with 6E10. M, D, and T represent the monomeric, dimeric, and trimeric forms, respectively. A longer exposure revealed a smear also in the fibrillized Aβ₁₋₄₀.

Intracellular Aβ₁₋₄₂ toxicity is selective to human neurons. The volume of primary cultured human astrocytes is 10 times that of human neurons; therefore, 100 niM of Aβ₁₋₄₂ was injected into the cytosol of astrocytes to keep the same concentration of injected Aβ. In human neurons, cell death is easily observed in the microinjected cells (red) by TUNEL (green) and condensed nuclear DNA (blue) (FIG. 3B).

In FIG. 3B, cells were microinjected with 100 nM Aβ₁₋₄₂ and DTR. Cell death was identified by TUNEL staining and nuclei detected with Hoechst staining. In contrast, microinjected astrocytes do not show any sign of condensed chromatin by Hoechst staining, nor positive nuclei TUNEL staining (FIG. 3B). The faint green fluorescence detected in these cells is the results of bleed-through from the red DTR fluorescence on the microscope.

Similarly, microinjections of the Aβ₁₋₄₂ peptide into human neuroblastoma, La—N-1 and M17 cell lines, teratocarcinoma NT2 cells, mice NIH 3T3 fibroblasts, and BHK cells fails to induce cell death. Quantitative analysis on 600 microinjected cells from 3 independent experiments confirms that among the cell lines tested, only human primary neurons are susceptible to the intracellular Aβ₁₋₄₂ toxicity.

Cell survival quantitation in several cell lines injected with Aβ₁₋₄₀, to Aβ₁₋₄₂ or Aβ₄₀₋₁, when compared with Aβ-injected and control DTR-injected cells, demonstrated that intracellular Aβ₁₋₄₂ is selectively toxic to human neurons.

Aβ₁₋₄₂ neurotoxicity requires de novo protein synthesis. To address the molecular mechanism of intracellular Aβ toxicity, Aβ₁₋₄₂ was microinjected into neurons and the cells were incubated in the presence or absence of transcriptional inhibitor actinomycin D and translational inhibitor cycloheximide for 24 hours. Both cycloheximide and actinomycin D efficiently block Aβ₁₋₄₂-induced neuronal death (FIG. 4).

FIG. 4 illustrates neuronal cell death in non- or Aβ₁₋₄₂-injected neurons incubated in the absence (−) or presence of 5 μg/ml cycloheximide (CHX) or 5 μM actinomycin D (ACTD) for 24 hours. Neurons were pre-incubated for 1 hour in CHX and ACTD before microinjections. These results indicate that de novo protein synthesis mediates intracellular Aβ toxicity.

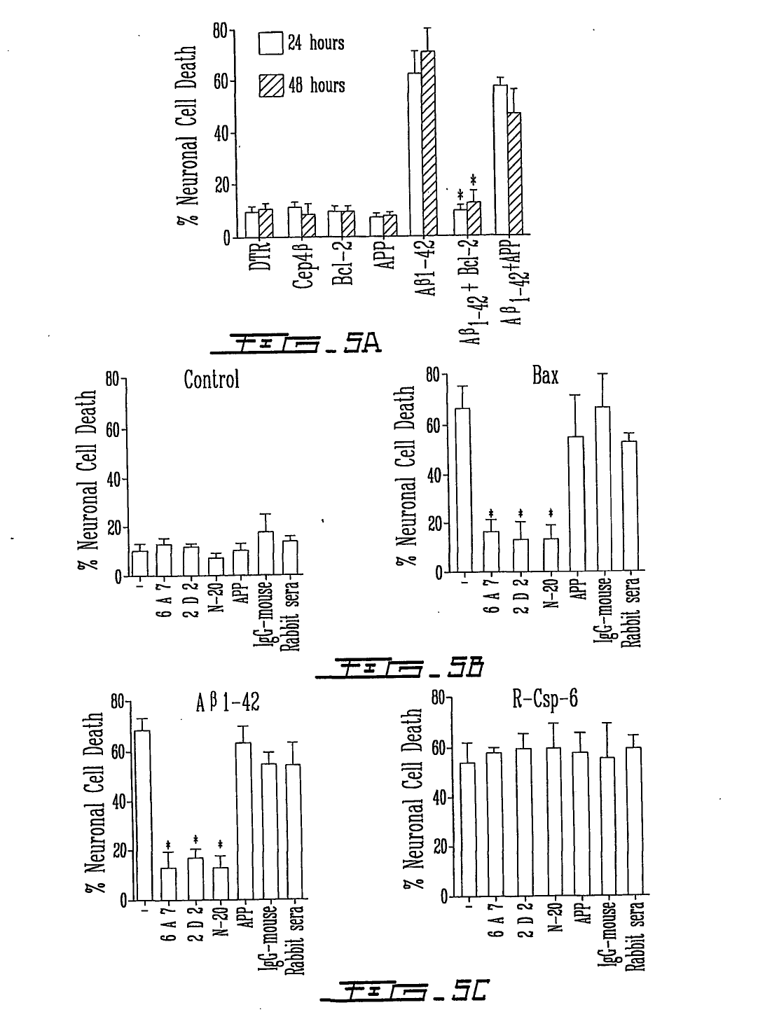

Bax may be responsible for intracellular Aβ toxicity. The activation of pro-apoptotic Bax can occur through transcriptional activation and because the rapidity with which Aβ₁₋₄₂ induces neuronal cell death is similar to that obtained through Bax-mediated neuronal apoptosis, it was suspected that Bax could be involved in this type of cell death. Therefore, a human Bcl-2 cDNA expression construct was microinjected with the Aβ₁₋₄₂ peptide (FIG. 5A).

FIG. 5A illustrates neuronal cell death in Aβ₁₋₄₂-microinjected neurons co-injected with a Bcl-2 or APP Cep4β eukaryotic cDNA expression episomal construct. The microinjection of the Bcl-2 construct completely eliminates Aβ₁₋₄₂ neurotoxicity as previously observed against Bax. In contrast, co-injection of an APP cDNA construct does not alter Aβ₁₋₄₂-mediated cell death.

To confirm that Bax is involved, antibodies against human Bax that could neutralize Bax-mediated killing were tested. It was found that two monoclonals antibodies, 6A7 and 2D2, and the polyclonal antisera N-20, but not an APP antibody, rabbit sera or mouse IgG, could neutralize the pro-apoptotic properties of Bax (FIG. 5B).

FIG. 5B illustrates neuronal cell death in Bax cDNA, Aβ₁₋₄₂ peptide, or recombinant active caspase-6 (R-Csp-6)-microinjected neurons in the absence or presence of monoclonal Bax antibodies, 6A7 or 2D2, Bax polyclonal antisera, N-20, APP monoclonal antibody 22C11, mouse IgG or rabbit non-immune sera. Similarly, these anti-Bax antibodies completely neutralized the Aβ₁₋₄₂-mediated neurotoxicity. In contrast, none of these antibodies had any effect on recombinant active caspase-6-mediated cell death. Therefore, these experiments show that anti-Bax antibodies specifically inhibit Bax- and Aβ₁₋₄₂-mediated cell death and indicate that Aβ₁₋₄₂ induces cell death through Bax activation. Together, these results implicate Bax in Aβ₁₋₄₂-mediated neuronal cell death.

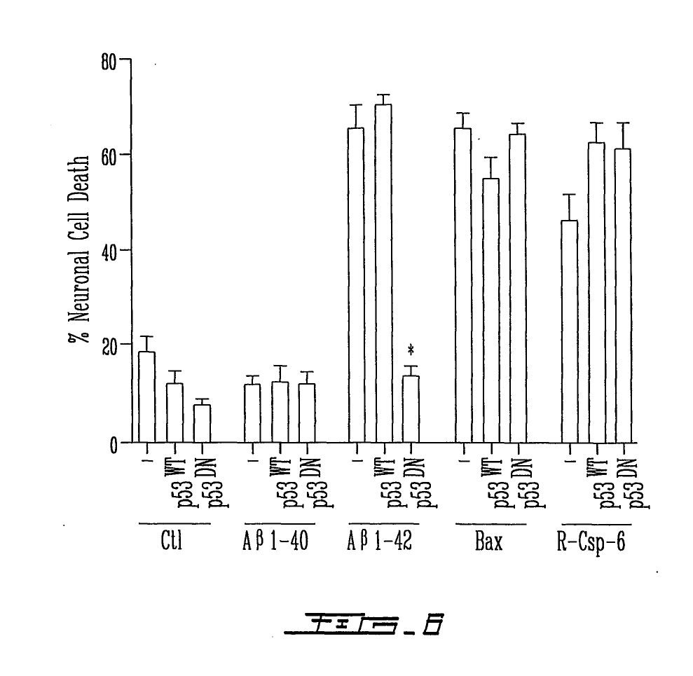

p53 is involved in intracellular Aβ₁₋₄₂mediated neurotoxicity. Because Bax is transcriptionally regulated by p53, the involvement of p53 activation in intracellular Aβ₁₋₄₂-mediated neurotoxicity was examined. The p53 R273H dominant negative (p53DN) mutant was chosen because it effectively inhibits p53 transcriptional activation of Bax (Aurelio, O. N., et al., Mol. Cell. Biol. 20:770-8, 2000). While the expression of p53 wild type or p53DN did not induce neuronal apoptosis in absence or presence of Aβ₁₁₄₀, the p53DN but not the p53 wild type, effectively inhibited Aβ₁₋₄₂-mediated neurotoxicity (FIG. 6).

FIG. 6 illustrates neuronal cell death in DTR only (DTR), Aβ₁₋₄₀ peptide, Aβ₁₋₄₂ peptide, R-Csp-6, or Bax cDNA co-microinjected with cDNA expressing wild type (WT) or dominant negative (DN) p53 in neurons. The inability of the p53DN to inhibit the toxicity of Bax when expressed from the CMV promoter of a cDNA construct or to inhibit cell death by recombinant active caspase-6 attests to the specificity of the p53DN against Aβ₁₋₄₂-mediated cell death. Together with the inhibition of Aβ₁₋₄₂-mediated neurotoxicity with Bax antibodies, these results show that Aβ₁₋₄₂ activates p53 which regulates Bax expression and cell death.

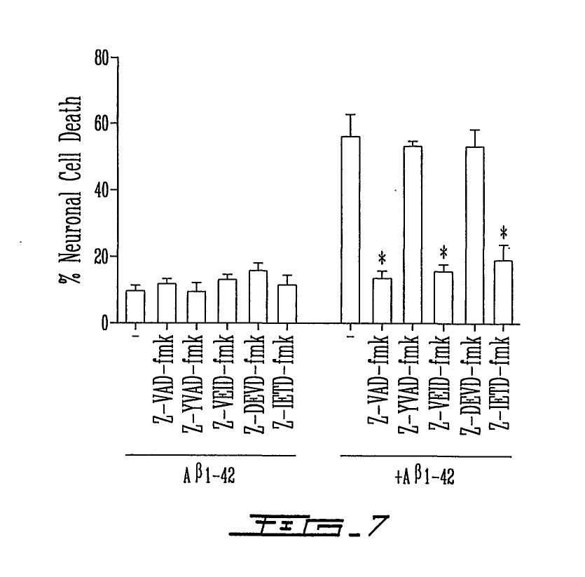

Caspase inhibitors prevent Aβ₁₋₄₂-mediated cell death. Previously, it has been shown that serum deprivation induces caspase-6 but not caspase-3-mediated cell death in human neurons (LeBlanc, A. C., et al., J. Biol. Chem. 274:23426-23436, 1999). To assess the role of caspases in Aβ₁₋₄₂-mediated neurotoxicity, Aβ₁₋₄₂-injected neurons were incubated in the presence or absence of various caspase inhibitors. The results show that the pan caspase inhibitor, Z-VAD-fmk, the caspase-6 inhibitor, Z-VEID-fmk, and the caspase-8 inhibitor, Z-IETD-fmk, completely prevents Aβ₁₋₄₂-induced neuronal cell death (FIG. 7).

In FIG. 7, neurons were pre-incubated for 1 hour in the presence of 5 μM of each inhibitor, microinjected with A₁₋₄₂ peptide and incubated for 24 hours in the presence of the inhibitors before revealing cell death in injected cells with TUNEL. In contrast, the caspase-1 inhibitor, Z-YVAD-fmk and the caspase-3 inhibitor, Z-DEVD-fmk only inhibit 30% of the Aβ₁₋₄₂-induced cell death. Therefore, the data show that caspase-6 or caspase-8-like enzymes regulate Aβ₁₋₄₂-mediated neuronal apoptosis.

The Aβ of AD senile plaques is generated intracellularly from APP in the endoplasmic-reticulum, trans-Golgi and endosomal-lysosomal system (Selkoe, D. J., Annu. Rev. Cell Biol. 10:373-403, 1994). Most of the Aβ is secreted rapidly and the accumulation of extracellular Aβ deposits in the senile plaques of AD has long been debated as a primary cause of AD.

The development of antibodies specific to the C-terminus of Aβ40 and Aβ42 has revealed the presence of intracellular Aβ42 in AD neurons (D'Andrea, M. R., et al., Histopathology 38:120-34, 2001; Gouras, G. K., et al., Am. J. Pathol. 156:15-20, 2000). The intraneuronal Aβ42 appears to precede the other pathological lesions of AD and raise the issue of whether intraneuronal Aβ42 is detrimental to neurons.

The data reported herein provide evidence that intracellular accumulation of Aβ₁₋₄₂ is a cause of neuronal loss in AD. Intracellular accumulation of Aβ₁₋₄₂ preceding other pathological lesions is observed not only in AD (D'Andrea et al., Supra; and Gouras et al., Supra) but in cells or transgenic brains expressing AD associated presenilin 1 mutations (Petanceska, S. S., et al., J. Neurochem. 74:1878-84, 2000; Sudoh, S., et al., J. Neurochem. 71:1535-43, 1998; and Xia, W., et al., Biochemistry 37:16465-71, 1998) and in aging monkeys (Martin, L., et al., American Journal ofpathology 145:1358-1381, 1994a).

Intracellular Aβ also increases in apoptotic human neurons and SH-SY5Y cells under oxidative stress (LeBlanc, A. C., and C. Goodyer., J. Neurochem. 72:1832-1842, 1999; and Misonou, H. et al., Biochemistry 39:6951-9, 2000). Despite most studies showing that extracellular Aβ deposition in senile plaques does not correlate well with the severity of AD, analysis of Aβ levels extracted from the brains of well ascertained cognitively impaired patients revealed an elevation of both Aβ40 and Aβ42 levels with increasing deficits (Naslund, J., et al., JAMA 283:1571-7, 2000). In the frontal cortex, the increased Aβ levels preceded significant neurofibrillary tangle pathology. Therefore, the data herein show that intracellular Aβ may be involved in cognitive decline and may in fact be the initial insult leading to neuronal dysfunction or death.

The data reported herein also show that Aβ₁₋₄₂ mediates neurotoxicity through the known p53 and Bax mediated cell death pathway. p53 expression is also increased in the cytosolic Aβ₁₋₄₂ transgenic brain (LaFerla, F. M., et al., J. Clin. Invest. 98:1626-32, 1996). In addition, synthetic p53 inhibitors can prevent Aβ mediated toxicity of hippocampal neuron cultures (Culmsee, C., et al., J. Neurochem. 77:220-8, 2001). Increased p53 levels have been observed in AD brains and there is also evidence for increased Bax protein levels in AD.

Further validation of intracellular toxicity of Aβ₁₋₄₂. Lithium inhibits selectively GSK3p, a kinase that is activated and directly involved in apoptosis. GSK3β is inhibited by a number of survival pathways including the Akt survival kinase linked to the signal transduction of neurotrophic factors (reviewed in Grimes CA and Jope RS Prog. Neurobiol. 65:391-426 (2001)). GSK3β when activated regulates negatively a number of transcription factors involved in neuronal survival. In addition, GSK3β can phosphorylate Tau leading to hyperphosphorylation and the appearance in cultures of the pathological PHF-1 marker of Tau. Furthermore, GSK3β has been implicated in extracellular Aβ toxicity in PC12 and rat primary cortical neurons cells (Alvarez G., et al., FEBS Lett. 453:260-264 (1999); Wei H., et al., Eur. J. Pharmacol. 392:117-123 (2000)). Therefore, to further define the signal transduction pathway involved in intracellular Aβ toxicity, the involvement of GSK3β was tested with lithium, a selective GSK3β inhibitor.

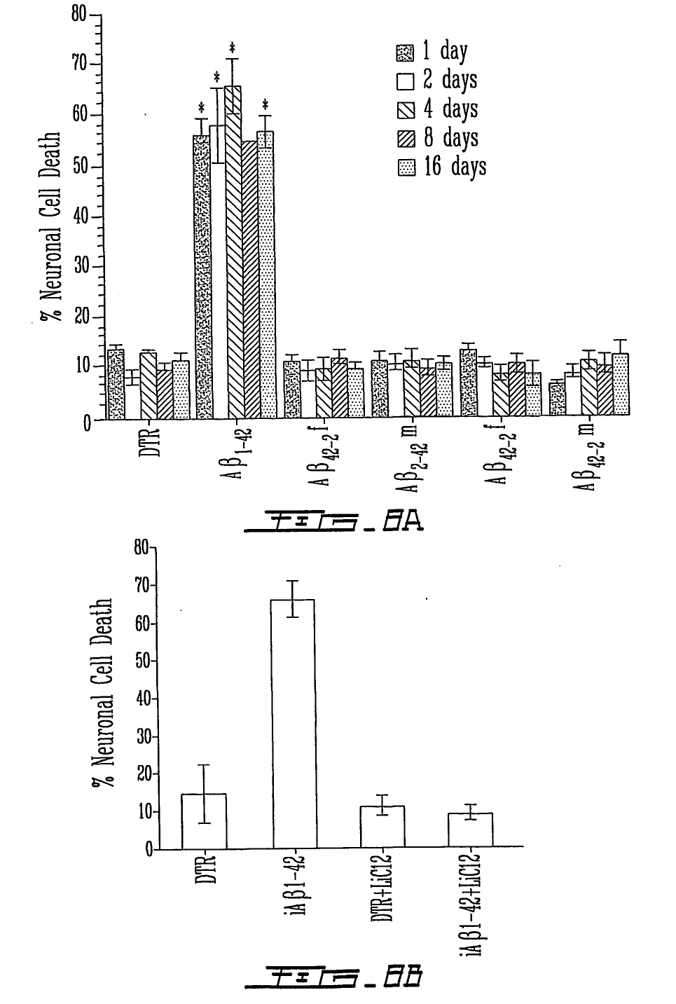

Neurons were pre-incubated with 20 mM LiCl₂ for 1 hour, microinjected with a lethal dose of intracellular Aβ₁₋₄₂, and incubated in the presence of the LiCl₂ for 24 hours before fixing the cells and performing TUNEL analyses to assess neuronal cell death, as illustrated in FIG. 8B. Dextran Texas Red (DTR) was co-injected with Aβ₁₋₄₂ to identi the injected cells from the non-injected cells. The experiment was done on 200 microinjected neurons per preparation of neurons and on three independent neuron preparations.

The results show that as described above, intracellular Aβ₁₋₄₂induces 60-70% neuronal cell death within 24 hours of microinjection. DTR alone or with LiCl₂ treatment did not undergo cell death. However, LiCl₂ efficiently protected the human neurons against intracellular Aβ₁₋₄₂ toxicity. Because lithium selectively inhibits GSK3β these results strongly indicate that GSK3β is implicated in intracellular Aβ toxicity.

Cellular apoptosis induced by intracellular injection of Aβ₁₋₄₂

Preparation of the amyloid peptides. Amyloid peptides Aβ₁₋₄₂ and Aβ₁₋₄₀ (Bachem) and Aβ₄₀₋₁ (Sigma) were dissolved at 25 μM in sterile distilled water and incubated at 37° C. for five (5) days. The peptides were frozen and dissolved at the desired concentration in PBS before use.

Microinjections and quantitation of cellular apoptosis. Microinjections and assessment of celldeath were done as described in Zhang et al., J. Neurosci. 20:8384-8389 (2000). Briefly, amyloid peptides were miroinjected with the fluorescent marker dye Dextran Texas red (DTR) and processed at the indicated time for TUNEL. TUNEL and DTR-positive cells were calculated for % cell death. Data represents a minimum of six hundred microinjected cells in three independent experiments. Over 80-90% of microinjected neurons and 50-60% of other cell types survive the microinjection.

Unexpectedly, Aβ₂₋₄₂ generated by caspase cleavage was not found to be neurotoxic. The results indicate that both the N-terminal amino acid and the two C-terminal amino acids may be required for intracellular toxicity. Aβ₂₋₄₂ may therefore be a useful tool to analyze either the toxic structure of the peptide or the mechanism by which it selectively activates a cell death pathway. The data herein show that aspartic acid at the N-terminus may be required for Aβ toxicity. In one embodiment of the present invention, Aβ₂₋₄₂ is used as a tool to aid in screening for inhibitors of Aβ toxicity, e.g., as a negative control.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims. All references cited herein are incorporated herein by reference in their entirety.

What is claimed:

1. A method for treating or preventing a disease state associated with amyloidosis, said method comprising administering to a subject a therapeutically effective amount of a compound for reducing the intracellular concentration of Aβ, such that said disease state associated with amyloidosis is treated or prevented.

2. The method of claim 1, wherein said disease state is Aβ production or accumulation associated with Alzheimer's disease, cerebral amyloid angiopathy, Down's Syndrome, or inclusion body myositis.

3. The method of claim 1, wherein the compound is an intracellular protease capable of eliminating Aβ or preventing accumulation of Aβ.

4. The method of claim 3, wherein said protease is induced into neurons causing said disease.

5. A transfected cell capable of expressing an agent capable of inducing into neurons an intracellular protease capable of eliminating Aβ or preventing accumulation of Aβ, said cell being otherwise when not transfected, not capable of expressing said protease.

6. A gene therapy for treating an Aβ amyloid-associated disease or condition, comprising administering an expression vector to a patient suffering from the Aβ amyloid-associated disease or condition, said vector coding under suitable conditions for an agent capable of inducing into neurons an intracellular protease capable of eliminating Aβ or preventing accumulation of Aβ.

7. A method for preventing or inhibiting amyloid production in a subject, said method comprising administering to a subject a therapeutically effective amount of a compound capable of reducing the intracellular concentration of Aβ, such that intracellular amyloid production or accumulation is prevented or inhibited.

8. A method for preventing, reducing, or inhibiting amyloid production in a subject, said method comprising administering to a subject a therapeutically effective amount of a compound capable of inhibiting Aβ intracellular accumulation, such that Aβ pproduction is prevented, reduced, or inhibited.

9. A method for modulating amyloid-associated damage to cells, comprising the step of administering a compound capable of reducing the intracellular concentration of Aβ, such that said amyloid-associated damage to cells is modulated.

10. A method for preventing, reducing, or inhibiting amyloid production in a subject, said method comprising administering to a subject a therapeutically effective amount of a compound capable of inhibiting β-secretase or γ-secretase, such that intracellular Aβ production is prevented, reduced, or inhibited.

11. A method for preventing cell death in a subject, said method comprising administering to a subject a therapeutically effective amount of a compound capable of preventing Aβ-mediated events that lead to cell death.

12. The method of claim 11, wherein the compound capable of preventing Aβ-mediated events that lead to cell death is a caspase inhibitor.

13. The method of claim 12, wherein the caspase inhibitor is a caspase-6 or caspase-8 inhibitor.

14. The method of claim 12, wherein the caspase inhibitor is selected from the group consisting of pan caspase inhibitor, Z-VAD-fmk, Z-VEID-fmk and Z-EITD-fmk.

15. A method for screening a potential useful compound for treating or preventing an Aβ amyloid-associated disease or condition, said method comprising the steps of administering to a cell a compound to be screened and measuring inhibition of cell death mediated by Aβ or measuring an intracellular concentration of Aβ, wherein an intracellular concentration of Aβ lower than a concentration of Aβ measured for a normal cell is indicative of said compound being useful for treating the Aβ amyloid-associated disease or condition.

16. A method for treating or preventing an Aβ amyloid-associated disease or condition, said method comprising the step of breaking down intracellular Aβ or causing excretion of Aβ for reducing the intracellular concentration of Aβ, thereby treating or preventing the Aβ amyloid-associated disease or condition.

17. A method for preventing Aβ-mediated neurotoxicity in a patient, said method comprising the step of administering to said patient an anti-apoptotic compound for inhibiting pro apoptotic properties of Bax.

18. The method of claim 17, wherein the anti-apoptotic compound is selected from the group consisting of humanized monoclonal antibodies and polyclonal antibodies.

19. A method for preventing Aβ-mediated neurotoxicity in a patient, said method comprising the step of inactivation of p53 pro-apoptotic pathway for inhibiting neurotoxicity of Aβ.

20. The method of claim 19, wherein the step of inactivation is carried out by administration to the patient of p53DN mutant, inactivating the p53 pro-apoptotic pathway.

21. A method for identifying a compound capable of treating an Aβ amyloid-associated disease or condition comprising assaying the ability of the compound to modulate APP nucleic acid expression or intracellular Aβ toxicity, thereby identifying a compound capable of treating an Aβ amyloid-associated disease or condition.

22. A method for modulating an Aβ amyloid-associated disease or condition in a subject comprising contacting a cell of the subject with an agent that modulates intracellular APP expression or Aβ toxicity, such that an Aβ amyloid-associated disease or condition is modulated.

23. The method of any one of claims 21 or 22, wherein intracellular APP expression or Aβ toxicity is decreased.

24. The method of any one of claims 21 or 22, wherein the Aβ amyloid-associated disease or condition is selected from the group consisting of Alzheimer's disease, cerebral amyloid angiopathy, Down's Syndrome, or inclusion body myositis.

25. The method of any one of claims 21 or 22, wherein the ability of the compound to modulate intracellular APP expression or Aβ activity is determined by detecting cellular viability.

26. A method for modulating cellular viability in a cell comprising contacting a cell with a compound capable of modulating APP nucleic acid expression or Aβ toxicity, thereby modulating cellular viability in the cell.

27. The method of any one of claims 21 or 22, wherein the cell is a neuronal cell.

28. The method of claim 27, wherein the cell is derived from a primate.

29. The method of claim 28, wherein the cell is derived from a human.

30. The method of any one of claims 21 or 22, wherein the modulator is a small molecule having a molecular weight under 2500 Daltons, peptide, antisense oligonucleotide, antisense peptide, enzyme, antibody or fragment thereof, ribozyme, or haptomer.

31. A method of identifying a compound capable of modulating APP nucleic acid expression or Aβ toxicity in a cell, comprising
a) contacting a cell with a compound; and
b) assaying the ability of the test compound to modulate the expression of APP nucleic acid or the toxicity of Aβ, thereby identifying a compound capable of modulating APP nucleic acid expression or Aβ toxicity in a cell.

32. A method of identifying a compound capable of treating or preventing an amyloid-associated disease or condition, comprising
a) contacting a cell with a compound; and
b) assaying the ability of the test compound to modulate the expression of APP nucleic acid or the toxicity of Aβ, thereby identifying a compound capable of treating or preventing an amyloid-associated disease or condition.

33. The method of any one of claims 31 or 32, further comprising a step of introducing Aβ into the cub.

34. The method of any one of claims 31 or 32, wherein the cell is a neuronal cell.

35. The method of claim 34, wherein the cell is derived from a primate.

36. The method of claim 35, wherein the cell is derived from a human.

37. The method of any one of claims 31 or 32, wherein the compound is a small molecule having a molecular weight under 2500 Daltons, peptide, antisense oligonucleotide, enzyme, antibody, ribozyme, or haptomer.

38. The method of claim 33, wherein said introducing step is microinjection, contacting the cell with a liposome containing Aβ, transfection with an oligonucleotide encoding APP or Aβ, contacting the cell with a conjugate of APP gene product or Aβ with a peptide carrier, electroporation, contacting the cell with calcium chloride, contacting the cell with a DNA or RNA encoding APP or Aβ, or contacting a cell with a viral vector.

39. The method of any one of claims 31 or 32, wherein the assaying step is an apoptosis assay.

40. The method of claim 39, wherein said apoptosis assay is selected from the group consisting of TUNEL, measuring activation of caspases, MIT, or WST-1.

41. The method of any one of claims 31 or 32, wherein the compound is a member of a combinatorial library.

42. The method of any claim herein, wherein said Aβ is Aβ₁₋₄₂.

43. The method according to any claim herein, wherein said Aβ amyloid-associated disease or condition or said disease state associated with amyloidosis is selected from the group consisting of Alzheimer's disease, cerebral amyloid angiopathy, Down's Syndrome, and inclusion body myositis.

44. The method according to claim 25, wherein the cell conditionally expresses Aβ.

45. The method according to claim 44, wherein the expression of Aβ is regulated by a tetracycline-inducible gene expression system.
